(19) United States
(12) Patent Application Publication
(10) Pub. No.: US 2006/0148674 A1
(43) Pub. Date: Jul. 6, 2006

(54) Therapeutic composition

(76) Inventor: Richard F. Luduena, San Antonio, TX (US)

Correspondence Address: NEEDLE & ROSENBERG, P.C., SUITE 1000, 999 PEACHTREE STREET, ATLANTA, GA, 30309-3915, US

(21) Appl. No.: 11/026,803

(22) Filed: Dec. 31, 2004

(51) Int. Cl. A61K 38/54; A61K 31/12

(52) U.S. Cl. 514/2; 514/690

(57) ABSTRACT

An anti-mitotic composition with a therapeutic index greater than 1.1, a clonogenic survival rate of less than 0.1 percent, and a binding affinity index for a class II, a class III, or a class V beta tubulin isotype of at least about 1.1.

### FIELD OF THE INVENTION

An anti-mitotic composition with a therapeutic index greater than 1.1, a clonogenic survival rate of less than 0.1 percent, and a binding affinity ratio for one or more specified beta isotypes of tubulin of at least about 1.1.

### BACKGROUND OF THE INVENTION

It is known that antimitotic drugs bind to many diverse sites on tubulin and microtubules. See, e.g., page 262 of Mary Ann Jordan et al.'s article on “Microtubules as a Target for Anticancer Drugs, Nature Reviews/Cancer, Volume 4, April 2004, pages 253-265. At page 261 of the Jordan et al. article, it is disclosed that “Among the most important unsolved questions about the antitumour activities of microtubule-targeted drugs concern the basis of their tissue specificities and the basis of the development of drug resistance to these agents. For example, it is not known why paclitaxel is so effective against ovarian, mammary and lung tumours but essentially ineffective against many other solid tumors, such as kidney or colon carcinomas and various sarcomas. Similarly, for the Vinca alkaloids, it is unclear why they are frequently most effective against haematological cancers, but often ineffective against many solid tumours. There are clearly many determinants of sensitivity and resistance to antimitotic drugs, both at the level of the cells themselves and at the level of the pharmacological accessibility of the drugs to the tumour cells.”

At page 262 of the Jordan et al. article, it is disclosed that “. . . cells also have many microtubule-related mechanisms that confer resistance or determine intrinsic sensitivity to antimitotic drugs . . . . Microtubule-polymer levels and dynamics are regulated by a host of factors, including expression of regulatory proteins, post-translational modifications of tubulin and expression of different tubulin isotypes. The levels of each of these isotypes differ among tissue and cell types, and there are numerous examples of changes in their levels that correlate with development of resistance to paclitaxel or Vinca alkaloids and other microtubule-targeted drugs . . . . ”

One of the problems with many of the prior art anti-mitotic drugs is that, at concentrations required to kill substantially all of the cancer cells in an organism, the drugs kill many normal cells.

It is an object of this invention to provide an antimitotic composition that is effective but substantially less toxic than prior art antimitotic compositions.

### SUMMARY OF THE INVENTION

In accordance with this invention, there is provided a composition comprised of a first anti-mitotic drug. The composition has a therapeutic index greater than 1.1, a clonogenic survival rate of less than 0.1 percent, and a binding affinity ratio for the beta II, beta III, and beta V isotypes of tubulin of at least about 1.1

### BRIEF DESCRIPTION OF THE DRAWINGS

The invention will be described by reference to the specification and the drawings, in which like numerals refer to like elements, and wherein:

FIG. 1 is a flow diagram of one process of the invention;

FIG. 2 is a flow diagram of another process of the invention; and

FIG. 3 is a flow diagram of yet another process of the invention.

FIG. 4 is a flow diagram of yet another process of the invention; and

FIG. 5 is a flow diagram of yet another process of the invention.

### DESCRIPTION OF THE PREFERRED EMBODIMENTS

In one preferred embodiment, the composition of this invention is comprised of an anti-tubulin agent. In the next section of this specification, several of the properties of tubulin and its isotypes will be discussed.

Tubulin, the subunit protein of microtubules, is an alpha/beta(α/β) heterodimer. See, e.g., an article by J. Bryan entitled “Are cytoplasmic microtubules heteropolymers?” Proc. Nat. Acad. Sci. USA 68, 1762-1766. Reference also may be had to an article by R. F. Luduena et al., “Structure of the tubulin dimer,” J. Biol. Chem. 252, 7006-7014.

The full amino acid sequences of alpha and beta were first determined in 1981 and found to be 41% identical. The existence of tubulin isotypes was confirmed in this same work. The amino acid sequences of the peptides, obtained from pig brain tubulin, showed heterogeneity at various positions, indicating that at least four forms of alpha and two forms of beta were expressed in pig brain, presumably encoded by different genes. Since that time genes for alpha and beta tubulin have been sequenced from a large number of eukaryotes. Many of these organisms contain multiple genes for alpha or beta, or both, generally encoding proteins of different amino acid sequence. In this specification, applicant will refer to these different proteins as isotypes of alpha or beta, meaning proteins encoded by different genes with different amino acid sequences. More recently, other very different forms of tubulin have been discovered, designated as gamma, delta, epsilon, zeta, eta, theta, iota, and kappa (γ, δ, ε, ζ, η, θ, ι, and κ). Still others may be waiting in the wings. Some related proteins have been observed in prokaryotes as well. The term isotype has also been applied to different forms of alpha or beta differing in their post-translational modifications. In this specification, applicant will restrict the term isotype to proteins encoded by different genes, differing in amino acid sequence.

The area of tubulin isotypes has been discussed in the prior art. Reference may be had, e.g., to U.S. Pat. Nos. 5,386,013; 5,656,438; 5,661,032; 5,830,662; 5,837,844; 5,846,763; 5,854,202; 5,871,939; 5,888,818; 5,977,311; 6,000,722 (tubulin promoter regulates gene expression in neurons); 6,162,810; 6,172,205; 6,210,905; 6,214,571; 6,235,527; 6,251,682; 6,306,615 (detection method for monitoring beta tubulin 6,309,876; 6,331,396; 6,335,170; 6.363.321; 6,423,824; 6,441,139; 6,444,870; 6,518,397; 6.518,401; 6,627,405; 6,541,509; 6,686,198; and the like. The entire disclosure of each of these United States patents is hereby incorporated by reference into this specification.

Tubulin isotypes are also discussed in the literature references. See, e.g., R. F Luduena, “Are tubulin isotypes functionally significant?”, Mol. Biol. Cell 4, 445-457 (1993); R. F. Luduena, “The different forms of tubulin: different gene products and covalent modifications,” Int. Rev. Cytol. 178, 207-275 (1998); and Q. Lu et al., “Structural and functional properties of tubulin isotypes,” Adv. Struct. Biol. 5, 203-207 (1998).

The existence of tubulin isotypes has been demonstrated in many organisms It is clear that organisms in every eukaryotic phylum exhibit multiple isotypes of both alpha and beta-tubulin. This is particularly true for the higher eukaryotes. Among the animals, in every case where multiple isotypes of alpha and beta have been searched for, they have been found, with the possible exception of the sea urchin Lytechinus, where a single alpha-tubulin gene was reported.

The beta-tubulin isotypes are well known in the prior art and are discussed, e.g., in published U.S. patent application Ser. No. 2004/0121351, the entire disclosure of which is hereby incorporated by reference into this specification. As is disclosed in this published patent application, “The conservation of structure and regulatory functions among the beta-tubulin genes in three vertebrate species (chicken, mouse and human) allowed the identification of and categorization into six major classes of beta-tubulin polypeptide isotypes on the basis of their variable carboxyterminal ends. The specific, highly variable 15 carboxyterminal amino acids are very conserved among the various species. Beta-tubulins of categories I, II, and IV are closely related differing only 2-4% in contrast to categories III, V and VI which differ in 8-16% of amino acid positions [Sullivan K. F., 1988, Ann. Rev. Cell Biol. 4: 687-716].”

As is also disclosed in published U.S. patent application Ser. No. 2004/121351, “. . . the expression pattern is very similar between the various species as can be taken from the following table [Sullivan K. F., 1988, Ann. Rev. Cell Biol. 4: 687-716] which comprises the respective human members of each class” In the table appearing at page 1 of this publication, it is indicated that the class I beta isotype is ubiquitous, the class II beta isotype occurs mostly in the brain, the class III beta isotype occurs exclusively in the brain, the class IVa beta isotype occurs exclusively in the brain, and the class IVb beta isotype is ubiquitous.

As is also disclosed in published U.S. patent application Ser. No. 2004/121351, “The C terminal end of the beta-tubulins starting from amino acid 430 is regarded as highly variable between the various classes. Additionally, the members of the same class seem to be very conserved between the various species . . . . As tubulin molecules are involved in many processes and form part of many structures in the eucaryotic cell, they are possible targets for pharmaceutically active compounds. As tubulin is more particularly the main structural component of the microtubules it may act as point of attack for anticancer drugs such as vinblastine, colchicine, estramustine and taxol which interfere with microtubule function. The mode of action is such that cytostatic agents such as the ones mentioned above, bind to the carboxyterminal end of the beta-tubulin which upon such binding undergoes a conformational change. For example, Kavallaris et al. [Kavallaris et al. 1997, J. Clin. Invest. 100: 1282-1293] reported a change in the expression of of specific beta-tubulin isotypes (class I, II, III, and IVa) in taxol resistant epithelial ovarian tumor. It was concluded that these tubulins are involved in the formation of the taxol resistance. Also a high expression of class III beta-tubulins was found in some forms of lung cancer suggesting that this isotype may be used as a diagnostic marker.”

Preferred Binding Sites of the Anti-Mitotic Composition

The composition of this invention contains at least one anti-mitotic agent. In one preferred embodiment, such composition contains at least two anti-mitotic agents.

It is known that many chemotherapeutic drugs effect their primary actions by inhibiting tubulin polymerization. Thus, as is disclosed in U.S. Pat. No. 6,162,930 (the entire disclosure of which is hereby incorporated by reference into this specification), “An aggressive chemotherapeutic strategy toward the treatment and maintenance of solid-tumor cancers continues to rely on the development of architecturally new and biologically more potent anti-tumor, anti-mitotic agents. A variety of clinically-promising compounds which demonstrate potent cytotoxic and anti-tumor activity are known to effect their primary mode of action through an efficient inhibition of tubulin polymerization (Gerwick et al.). This class of compounds undergoes an initial binding interaction to the ubiquitous protein tubulin which in turn arrests the ability of tubulin to polymerize into microtubules which are essential components for cell maintenance and cell division (Owellen et al.).”

U.S. Pat. No. 6,162,930 also discloses that the precise means by which the cytotoxic agents “. . . arrests the ability of tubulin to polymerize . . . ” is unknown, stating that: “Currently the most recognized and clinically useful tubulin polymerization inhibitors for the treatment of cancer are vinblastine and vincristine (Lavielle, et al.). Additionally, the natural products rhizoxin (Nakada, et al., 1993a and 1993b; Boger et al.; Rao et al., 1992 and 1993; Kobayashi et al., 1992 and 1993) combretastin A-4 and A-2 (Lin et al.; Pettit, et al., 1982, 1985, and 1987) and taxol (Kingston et al.; Schiff et al; Swindell, et a, 1991; Parness, et al.) as well as certain synthetic analogues including the 2-styrylquinazolin-4(3H)-ones (SQO) (Jiang et al.) and highly oxygenated derivatives of cis- and trans-stilbene (Cushman et al.) and dihydrostilbene are all known to mediate their cytotoxic activity through a binding interaction with tubulin. The exact nature of this interaction remains unknown and most likely varies somewhat between the series of compounds.”

U.S. Pat. No. 6,512,003 also discusses the “. . . nature of this unknown interaction . . . ,” stating that (at column 1) “Novel tubulin-binding molecules, which, upon binding to tubulin, interfere with tubulin polymerization, can provide novel agents for the inhibition of cellular proliferation and treatement of cancer.” U.S. Pat. No. 6,512,003 presents a general discussion of the role of tubulin in cellular proliferation, disclosing (also at column 1) that: Cellular proliferation, for example, in cancer and other cell proliferative disorders, occurs as a result of cell division, or mitosis. Microtubules play a pivotal role in mitotic spindle assembly and cell division . . . . These cytoskeletal elements are formed by the self-association of the alpha/beta tubulin heterodimers . . . . Agents which induce depolymerization of tubulin and/or inhibit the polymerization of tubulin provide a therapeutic approach to the treatment of cell proliferation disorders such as cancer. Recently, the structure of the .alpha/beta tubulin dimer was resolved by electron crystallography of zinc-induced tubulin sheets . . . . According to the reported atomic model, each 46×40×65.ANG. tubulin monomer is made up of a 205 amino acid N-terminal GTP/GDP binding domain with a Rossman fold topology typical for nucleotide-binding proteins, a 180 amino acid intermediate domain comprised of a mixed 13 sheet and five helices which contain the taxol binding site, and a predominantly helical C-terminal domain implicated in binding of microtubule-associated protein (MAP) and motor proteins . . . . ”

Published U.S. patent application Ser. No. 2004/0044059, the entire disclosure of which is hereby incorporated by reference into this specification, also discloses the uncertainty that exists with regard to the “. . . tubulin binding site interactions . . . . ” At page 1 thereof, it states that: “The exact nature of tubulin binding site interactions remain largely unknown, and they definitely vary between each class of Tubulin Binding Agent. Photoaffinity labeling and other binding site elucidation techniques have identified three key binding sites on tubulin: 1) the Colchicine site (Floyd et al, Biochemistry, 1989; Staretz et al, J. Org. Chem., 1993; Williams et al, J. Biol. Chem., 1985; Wolff et al, Proc. Natl. Acad. Sci. U.S.A., 1991), 2) the Vinca Alkaloid site (Safa et al, Biochemistry, 1987), and 3) a site on the polymerized microtubule to which taxol binds (Rao et al, J. Natl. Cancer Inst., 1992; Lin et al, Biochemistry, 1989; Sawada et al, Bioconjugate Chem, 1993; Sawada et al, Biochem. Biophys. Res. Commun., 1991; Sawada et al, Biochem. Pharmacol., 1993). An important aspect of this work requires a detailed understanding, at the molecular level, of the ‘small molecule’ binding domain of both the α and β subunits of tubulin. The tertiary structure of the alpha/beta tubulin heterodimer was reported in 1998 by Downing and co-workers at a resolution of 3.7 Å using a technique known as electron crystallography (Nogales et al, Nature, 1998). This brilliant accomplishment culminates decades of work directed toward the elucidation of this structure and should facilitate the identification of small molecule binding sites, such as the colchicine site, using techniques such as photoaffinity and chemical affinity labeling (Chavan et al, Bioconjugate Chem., 1993; Hahn et al, Photochem. Photobiol., 1992).”

The Anti-Mitotic Activity of the Composition of this Invention

The composition of this invention is comprised of at least one anti-mitotic agent and, more preferably, at least two such anti-mitotic agents. Each of such anti-mitotic agents preferably has a mitotic index factor of at least about 10 percent and, more preferably, at least about 20 percent. In one aspect of this embodiment, the mitotic index factor of each of such anti-mitotic agents is at least about 30 percent. In another embodiment, the mitotic index factor of each of these agents is at least about 50 percent.

As is known to those skilled in the art, the mitotic index is a measure of the extent of mitosis. Reference may be had, e.g., to U.S. Pat. Nos. 5,262,409 (binary tumor therapy), 5,443,962 (methods of identifying inhibitors of cdc25 phosphatase), 5,744,300 (methods and reagents for the identification and regulation of senescence-related genes), 6,613,318, 6,251,585 (assay and reagents for identifying anti-proliferative agents), 6,252,058 (sequences for targeting metastatic cells), 6,387,642 (method for identifying a reagent that modulates Myt1 activity), 6,413,735 (method of screening for a modulator of angiogenesis), 6,531,479 (anti-cancer compounds), 6,599,694 (method of characterizing potential therapeutics by determining cell-cell interactions), 6,620,403 (in vivo chemosensitivity screen for human tumors), 6,699,854 (anti-cancer compounds), 6,743,576 (database system for predictive cellular bioinformatics), and the like. The entire disclosure of each of these United States patents is hereby incorporated by reference into this specification.

Reference may also be had, e.g., to U.S. Pat. No. 5,262,409, which discloses that: “Determination of mitotic index: For testing mitotic blockage with nocodazole and taxol, cells were grown a minimum of 16 hours on polylysine-coated glass coverslips before drug treatment. Cells were fixed at intervals, stained with antibodies to detect lamin B, and counterstained with propidium iodide to assay chromosome condensation. To test cell cycle blocks in interphase, cells were synchronized in mitosis by addition of nocodazole (Sigma Chemical Co.) to a final concentration of 0.05 μg/ml from a 1 mg/ml stock in dimethylsulfoxide. After 12 hours arrest, the mitotic subpopulation was isolated by shakeoff from the culture plate. After applying cell cycle blocking drugs and/or 2-AP, cells were fixed at intervals, prepared for indirect immunofluorescence with anti-tubulin antibodies, and counterstained with propidium iodide. All data timepoints represent averages of three counts of greater than 150 cells each. Standard deviation was never more than 1.5% on the ordinate scale.”

Reference may be had, e.g., to U.S. Pat. No. 6,413,735 which discloses that: “The mitotic index is determined according to procedures standard in the art. Keram et al., Cancer Genet. Cytogenet. 55:235 (1991). Harvested cells are fixed in methanol:acetic acid (3:1, v:v), counted, and resuspended at 106 cells/ml in fixative. Ten microliters of this suspension is placed on a slide, dried, and treated with Giemsa stain. The cells in metaphase are counted under a light microscope, and the mitotic index is calculated by dividing the number of metaphase cells by the total number of cells on the slide. Statistical analysis of comparisons of mitotic indices is performed using the 2-sided paired t-test.”

By means of yet further illustration, one may measure the mitotic index by means of the procedures described in, e.g., articles by Keila Torres et al. (“Mechanisms of Taxol-Induced Cell Death are Concentration Dependent,” Cancer Research 58, 3620-3626, Aug. 15, 1998), and Jie-Gung Chen et al. (“Differential Mitosis Responses to Microtubule-stabilizing and destabliling Drugs,” Cancer Research 62, 1935-1938, Apr. 1, 2002).

The mitotic index is preferably measured by using the well-known HeLa cell lines. As is known to those skilled in the art, HeLa cells are cells that have been derived from a human carcinoma of the cervix from a patient named Henrietta Lack; the cells have been maintained in tissue culture since 1953.

Hela cells are described, e.g., in U.S. Pat. Nos. 5,811,282 (cell lines useful for detection of human immunodeficiency virus), 5,376,525 (method for the detection of mycoplasma), 6,143,512, 6,326,196, 6,365,394 (cell lines and constructs useful in production of E-1 deleted adenoviruses), 6,440,658 (assay method for determining effect on adenovirus infection of Hela cells), 6,461,809; 6,596,535, 6,605,426, 6,610,493 (screening compounds for the ability to alter the production of amyloid-beta-peptide), 6,699,851 (cytotoxic compounds and their use), and the like; the entire disclosure of each of these United States patents is hereby incorporated by reference into this specification. By way of illustration, U.S. Pat. No. 6,440,658 discloses that, for the experiments described in such patent, “The HeLa cell line was obtained from the American Type Culture Collection, Manassas Va.”

In one preferred embodiment, the mitotic index of a “control cell line” (i.e., one that omits that drug to be tested) and of a cell line that includes 50 nanomoles of such drug per liter of the cell line are determined and compared. The “mitotic index factor” is equal to (Mt−Mc/Mc)×100, wherein Mc is the mitotic index of the “control cell line,” and Mt is the mitotic index of the cell line that includes the drug to be tested.

Presence of the Beta Tubulin Isotypes in Mammalian Tissues.

The beta I tubulin isotype appears to be the most widespread among mammalian tissues. It has been seen in almost every tissue that has been examined. It is also found in many avian tissues. It is extremely highly conserved in evolution: although the avian and mammalian lines diverged 310 million years ago, chicken and mouse beta I are identical in all 444 residues. The relative amounts of beta I in different tissues are very variable.

The beta II tubulin isotype has been widely studied. The brain is the source of the tubulin used in the vast majority of experimentation in vitro. Since beta II constitutes 58 percent of the total beta-tubulin in bovine brain, it appears that beta II is the best studied of the tubulin isotypes. For this reason, it is highly ironic that so little is known about beta II's specific function. However, since beta II is highly conserved in evolution, it probably has a particular role to play.

Beta II has a considerably more restricted distribution than does beta I. Beta II is prominent in the brain, where it is expressed in both neurons and glia. Beta II is also found in skeletal and smooth muscle and in connective tissue). It is found in the breast, adrenal and testes as well. In other tissues where beta II occurs, it is more likely to be restricted to a single cell type than is beta I. For example, in the skin, where beta I is expressed in each of the three layers of the stratum malpighii, beta II is concentrated in only one of these layers, the stratum granulosum.

Beta II is more widespread in early development. In fetal rats, not only does beta II occur in muscles, nerves and connective tissue but also in the retina, chondrocytes and endothelial cells. Not surprisingly, beta II also is found in neural stem cells. Unlike beta I and beta IV, beta II is generally not associated with axonemal microtubules except for those of the cilia of olfactory epithelia.

A highly unusual property of beta II has recently been discovered. Ranganathan et al. (“Immunohistochemical analysis of beta-tubulin isotypes in human prostate carcinoma and benign prostatic hypertrophy,” Prostate 30, 263-268, 1997) observed that beta II, but not beta I, beta III or beta IV, occurred in the cell nuclei of prostate tumors and benign prostate hyperplasia. A later study showed that beta II was present in the nuclei of cultured rat kidney mesangial cells in interphase (C. Walss et al., “Presence of B_{II}-isotype of tubulin in the nuclei of cultured rat kidney mesangial cells,” Cell. Motil. Cytoskeleton 42, 274-284, 1999). In these cells, an antibody to beta II strongly stained the nuclei but not the cytoplasm. The staining occurred throughout the nuclei, but was concentrated in the nucleoli. When the mesangial cells enter mitosis, the beta II leaves the nuclei and helps to form the mitotic spindle. During telophase, beta II enters the re-forming nucleus. In contrast, in these cells, beta I and beta IV, that constitute the interphase nicrotubule network, enter the spindle during mitosis, at the end of mitosis returning to the interphase network. These two isotypes never enter the nuclei.

Other studies revealed that only certain cultured non-transformed cells contained nuclear beta II, whereas nuclear beta II occurred in almost every cultured cancer cell (C. Walss-Bass et al., “Occurrence of nuclear beta II-tubulin in cultured cells, “Cell and Tissue Research, 308, 215-223, 2002). A survey of about 200 tumors excised from patients showed nuclear bII in 74% of them (I.-T. Yeh et al., “The beta II isotype of tubulin is present in the cell nuclei of a variety of cancers,” Cell Motil. Cytoskeleton 57, 96-106, 2004). In general, nuclear beta II staining was very variable, depending on the tumor type. In tumors of the prostate, stomach, and colon, nuclear beta II was seen in every sample studied. In contrast, only a few hepatic and brain tumors showed nuclear beta II. In some excisions, nuclear beta II occurred in almost every tumor cell, but sometimes in only a fraction. The intensity of nuclear staining also varied. The pattern of intra-nuclear staining was variable as well. In some cases, beta II was concentrated in the nucleoli; in others it appeared to stain the entire nucleoplasm except the nucleoli. Cytoplasmic staining of beta II was also highly variable. Many samples appeared to have beta II only in their nuclei and not in the cytoplasm.

The study with human tumors revealed two unusual patterns. First, nuclear beta II occurred in tumors of tissues, such as the prostate, in which the normal tissue does not express beta II. This would suggest that transformation leads cells first to express beta II and then to localize it to the nuclei. Second, otherwise normal cells near the tumor would also contain nuclear beta II. This was particularly striking in cases of breast cancers that had metastasized to the lymph nodes. Lymphocytes normally do not stain for beta II. However, lymphocytes adjacent to the metastatic cancer cells contained nuclear beta II. These results suggest that a cancer cell can influence adjacent normal cells to make beta II and put it in the nuclei. Analysis of a number of normal tissues indicated that most of them did not contain nuclear beta II The exceptions were bone marrow, placenta, and pancreatic acinar cells.

The beta III isotype has six distinguishing characteristics, each of which is probably relevant to developing an understanding of its functional significance.

Beta III highly conserved in evolution. As is the case with beta I, there are only two differences in the amino acid sequences of chicken and human beta III. Beta III has a highly unusual distribution of cysteines. All the vertebrate beta isotypes have cysteines at positions 12, 127, 129, 201, 211, 303 and 354. The more widely distributed beta isotypes—beta I, beta II and beta IV—also have a cysteine at position 239. Beta III lacks this cysteine but has a cysteine at position 124 instead, where beta I, beta II and beta IV have a serine.

Beta III has an extremely narrow distribution in normal adult tissues. It is most abundant in the brain, where it is found only in neurons and not in glial cells (by contrast, beta II is found in both). Its absence from glial cells has made beta III a useful marker for neuronal differentiation. Beta III synthesis can be induced by factors such as androgens, and nerve growth factor. The latter, when combined with retinoic acid, can cause human umbilical cord blood cells to synthesize beta III as well as other neuronal proteins. Beta III also occurs in Sertoli cells and, in small amounts, in the vestibular organ, the nasal epithelia, and the colon. In other adult tissues that have been examined, beta III appears to be absent. However, beta III is found in a large number of cancers and is also widespread in some developing tissues. Beta III accounts for 25 percent of the total beta-tubulin in the brains of cows and 20 percent in deer brains.

Many tumors express beta III, including some of non-neuronal origin, such as lymphomas. Reference may be had, e.g., two articles by C. D. Katsetos et al. “Class III beta-tubulin isotype . . . ” (J. Child Neurol. 18, 851-866, 2003), and “Class II beta-tubulin in human development and cancer,” Cell Motil. Cytoskeleton 55, 77-96, 2003).

It has been observed that tumors of higher malignancy express higher levels of beta III. A study of patients with non-small cell lung cancer showed that those whose tumors had elevated beta III responded less well to drugs and had a poorer prognosis (see, e.g., an article by C. Dumontet et al., “Expression of class III beta-tubulin in non-small cell lung cancer is correlated with resistance to taxane chemotherapy, Elect. J. Oncol. 1, 58-64, 2002). Applicant has discovered that, when he compared the MCF-7 and BT-549 breast cancer cell lines, the latter, which has much more beta III than the former, and is resistant to taxol, vinblastine, and cryptophycin, also had a much higher level of free radicals.

The thioredoxin system is a set of proteins that cells use to protect themselves from free radicals. As is disclosed in U.S. Pat. No. 6,566,514, the entire disclosure of which is hereby incorporated by reference into this specification, “Thioredoxin is a small ubiquitous redox protein originally identified as a reducing cofactor for ribonucleotide reductase which is essential for DNA synthesis . . . . Thioredoxin and thioredoxin reductase comprise the thioredoxin system. Thioredoxin reductase is a selenocysteine containing flavoenzyme which uses NADPH as a proton donor to reduce thioredoxin which in turn reduces other proteins and therefore influences their functions.”

U.S. Pat. No. 6,566,514 also discloses that “In recent years, mammalian thioredoxin has been implicated in a variety of other biochemical pathways. For example, it modulates redox properties of transcription factors by dithiol disulfide exchanges, which alter their DNA binding characteristics. Transcription factors such as NF-.kappa.B3, BZLFI4 and TFIIIC5 are directly regulated, while AP-1 activation is mediated indirectly through the nuclear redox factor . . . which is further reduced by thioredoxin . . . In addition, thioredoxin has been shown to facilitate refolding of disulfide-containing proteins, to activate the glucocorticoid or interleukin-2-receptors, to inhibit human immunodeficiency virus expression in macrophages, to reduce H₂ O₂ scavenge free radicals, to protect cells against oxidative stress and to be an early pregnancy factor . . . . Cloned human thioredoxin has been shown to be similar to a growth factor termed adult T-cell leukemia-derived factor, released by HTLV-1 transformed T cells . . . . It utilizes a pathway for secretion. Extracellularly expressed thioredoxin stimulates the proliferation of normal fibroblasts, lymphoid cells and a number of human solid tumor cell lines . . . . Redox-inactive forms have been used to show that the growth stimulation requires a redox activity of thioredoxin . . . . The growth stimulation by thioredoxin appears to be induced indirectly through sensitizing cells to other growth factors . . . . Subsequently, thioredoxin has been reported to be over-expressed in some primary tumors such as lung, colon, cervical and hepatocellular carcinoma . . . Furthermore, human breast cancer cells transfected with wild-type thioredoxin cDNA have shown increased tumor growth . . . , decreased spontaneous apoptosis in vivo . . . and decreased sensitivity to apoptosis induced by a variety of anticancer therapeutic compounds . . . . On the other hand, cells transfected with dominant-negative, redox-inactive mutant thioredoxin have shown reduced anchorage-independent growth in vitro and inhibition of tumor growth in vivo . . . . ”.

U.S. Pat. No. 6,566,514 also discloses that “Thioredoxin reductase has been shown to be overexpressed by a number of human tumors . . . . Inhibition of cellular thioredoxin reductase by antitumor quinines . . . , nitrosoureas . . . and 13-cis-retinoic acid . . . have led to a decreased activity of the thioredoxin system and consequent contribution to the growth inhibitory activity . . . . ”

Mammals have two forms of beta IV, designated as beta IVa and beta IVb. The former is expressed only in the brain, while the latter is expressed in many tissues including the brain. The sequence differences between the two are minor, always involving very conservative amino acid substitutions.

Beta IV has one very clear-cut function: it occurs in axonemes. In mammals, beta IV has been localized in sperm flagella and in cilia of the tracheal epithelium, brain ependyma, oviduct, efferent duct of the testis, vestibular hair cells, retinal rod cells, olfactory neurons, and esophageal progenitor cells. In fact, beta IV has been found in every mammalian axoneme that has been tested.

Beta V is the most intriguing of the beta isotypes. It is highly conserved in evolution, suggesting that it may have a specific function. However, not only is that function unknown, we do not even know the normal distribution of beta V. Using mRNA measurements, Sullivan et al. showed that in chickens beta V is found in every tissue outside of the brain. Preliminary results with a monoclonal antibody to beta V, however, suggest that it is found in mammalian brain but in relatively few other tissues.

Perhaps the only clue to the function of beta V is that it has the same distribution of cysteine residues as beta III. In other words, it has cys124 but lacks cys239.

There is not much to say about the specific functions, if any, of the a isotypes in mammals. Their tissue distributions seem much less complex, as far as is known, than the distributions of the beta isotypes, Alpha I is found mostly in brain but also in a variety of other tissues. Alpha 3/7 is found only in the testis, where it is the major alpha isotype. Alpha 4 is widespread, especially in muscle and heart; alpha 6 is also widespread, but less common than the others. Alpha 8 is considerably divergent in sequence, being only 89% identical to the other alpha's (except for the even more divergent aTTI); it is found in heart, testis, and skeletal muscle, and at very low levels in the brain and pancreas. Alpha TT1 is even more divergent and is found only in the testis, where it is a minor component of the a population.

The Suppressivity of the Anti-Mitotic Composition

In preferred embodiment, the composition of this invention is comprised of at least one anti-tubulin agent that has a suppressivity of at least about 1000. As used in this specification, the term suppressivity refers to the ability of the anti-tubulin agent to suppress microtubule dynamic behavior. Reference may be had, e.g., to an article by W. B. Derry et al., “Taxol differentially modulates the dynamics of microtubules assembled from unfractionated and purified beta-tubulin isotypes,” Biochemistry, 36, 3554-3562.

Suppressivity is described by the equation Sp=|(y−b)/x|, wherein Sp is the suppressivity, y is the microtubule shortening rate in the presence of the drug being tested expressed as a percentage of the microtubule shortening rate in the absence of the drug, b the microtubule shortening rate in the absence of the drug being tested, and x the number of moles of the drug being tested bound to each mole of tubulin in the microtubule. The Derry et al. article discloses a process for determining the microtubule shortening rate both in the presence of the drug being tested and in its absence. Reference also may be had to U.S. Pat. No. 6,660,767 which describes microtubule shortening. Reference also may be had to U.S. published patent application 2002/0151560, the entire disclosure of which is hereby incorporated by reference into this specification.

In one embodiment, the anti-mitotic agent has a suppressivity of at least 1,000 with regard to a tubulin isotype selected from the group consisting of the class III isotype of beta tubulin and the class V isotype of beta tubulin. Note that in describing suppressivity, we are discussing a measurement done on a microtubule, not on a molecule of that isotype. The microtubule in question is made of tubulin alpha/beta dimers whose beta subunit is isotypically pure, i.e., the beta subunit is either beta-I, beta-II, beta-III, beta-IVa, beta-IVb, beta-V, beta-VI, or beta-VII; in contrast, the alpha subunit can be any one of the various alpha isotypes expressed in normal or cancerous cells and tissues and the microtubule being tested could contain any or all of these alpha isotypes. In one aspect of this embodiment, the suppressivity of the anti-mitotic agent with regard to the beta-III or beta-V isotypes is at least as great as it is with regard to class II or class IV.

One may determine the suppressivity of the antimitotic agent with regard to any particular beta-tubulin isotype in accordance with the process of the aforementioned Derry et al. paper. In particular, one may purify the particular tubulin isotype in question and, thereafter, test the suppressivity of the candidate drug against it.

A Compositon Comprised of a Thioredoxin System Inhibitor

In one preferred embodiment, the composition of this invention contains a thioredoxin system inhibitor.

Thioredoxin is a heat-stable protein that can exist as a dithiol, thioredoxin-(SH)₂, or as a disulfide, thioredoxin-S₂, and that serves to reduce ribonucleoside diphosphates to deoxyribonucleoside diphosphates. Thioredoxin reductase is the enzyme that catalyzes the reduction of thioredoxin-S₂ to thioredoxin-(SH)₂ with the oxidation of NADPH to NADP⁺; see, e.g., page 482 of J. Stensch's “Dictionary of Biochemistry and Molecular Biology,” Second Edition (John Wiley & Sons, New York, N.Y., 1989).

Thioredoxin, and the thioredoxin system, are well known in the art. Reference may be had, e.g., to U.S. Pat. Nos. 4,738,841 (use of thioredoxin, thioredoxin-derived, or thioredoxin-like dithiol peptides in hair care preparations), 4,849,223 (use of thioredoxin, thioredoxin-derived, or thioredoxin-like dithiol peptides in hair care preparations), 4,849,346 (method for determining thioredoxin reductase activity), 4,904,602 (thioredoxin shufflease and use thereof), 4,919,924 (use of thioredoxin, thioredoxin-derived, or thioredoxin-like dithiol peptides in hair care preparations), 4,935,231 (use of thioredoxin, thioredoxin-derived, or thioredoxin-like dithiol peptides in hair care preparations), 4,941,885 (hair removing composition and method containing thioredoxin, thioredoxin-derived, and thioredoxin-like peptides), 5,028,419 (use of thioredoxin, thioredoxin-derived, or thioredoxin-like dithiol peptides in hair care preparations), 5,270,181 (peptide and protein fusions to thioredoxin and thioredoxin-like molecules), 5,292,646 (peptide and protein fusions to thioredoxin and thioredoxin-like molecules), 5,646,016 (use of thioredoxin, thioredoxin-derived, or thioredoxin-like dithiol peptides in hair care preparations), 5,792,506 (neutralization of food allergens by thioredoxin), 5,8321,049 (human thioredoxin), 5,919,657 (nucleic acids encoding human thioredoxin protein), 5,952,034 (increasing the digestibility of food proteins by thioredoxin reduction), 5,985,261 (use of thioredoxin-like molecules for induction of MnSOD to treat oxidative damage), 6,143,524 (use of thioredoxin, thioredoxin-derived, or thioredoxin-like dithiol peptides in hair care preparations), 6,150,135 (dirofilaria and brugia thioredoxin peroxidase type-2 (TPX-2) nucleic acid molecules), 6,174,682 (thioredoxin family active site molecules and uses therefore), 6,190,723 (neutralization of food allergens by thioredoxin), 6,352,836 (dirofilaria and brugia thioredoxin peroxidase tpe-2 proteins), 6,380,372 (barley gene for thioredoxin and NADP-thioredoxin reductase), 6,462,187 (22109, a novel human thioredoxin family member), 6,555,116 (alleviation of the allergenic potential of airborne and contact allergens by thioredoxin), 6,566,514 (oligonucelotide sequences complemetary to thioredoxin or thioredoxin reductase genes and methods of using same to modulate cell growth), 6,689,775 (uses of thioredoxin), 6,750,046 (preparation of thioredoxin and thioredoxin reductase proteins on oil bodies), 6,767,536 (recombinant staphylococcus thioredoxin reductase and inhibitors therefore), 6,784,346 (value-added traits in grain and seed transformed with thioredoxin), and the like. The entire disclosure of each of these United States patents is hereby incorporated by reference into this specification.

The term thioredoxin system, as used in this specification, refers to a system which contains the small protein thioredoxin, the enzyme thioredoxin reductase, and the coenzyme NADPH (nicotinamide adenine dinucleotide phosphate) Reference may be had, e.g., to U.S. Pat. Nos. 5,792,506 (neutralization of food allergens by thioredoxin), 6,190,723 (neutralization of food allergens by thioredoxin), 6,113,951 and 6,114,504 (use of thiol redox proteins for reducing protein intramolecular disulfide bonds), for improving the quality of cereal products, dough, and baked goods, and for inactivating snake, bee, and scorpion toxins), 6,326,184, 6,372,772 (inhibitors of redox signaling), 6,552,060, 6,555,116, 6,610,334, 6,660,021, 6,750,046; 6,767,536 (Recombinant Staphyloccus thioredoxin reductase and inhibitors thereof), and the like. The entire disclosure of each of these United States patents is hereby incorporated by reference into this specification.

In one preferred embodiment, the composition of this invention is comprised of a thioredoxin system inhibitor. This type of inhibitor is well known in the art. Reference may be had, e.g., to the S. aureus thioredoxin reductase inhibitors disclosed in published United patent applications 2003/0166843 and 2004/0161809; the entire disclosure of each of these published United States patent applications is hereby incorporated by reference into this specification.

Reference may be had, e.g., to U.S. Pat. No. 6,372,772 (“Inhibitors of redox signaling . . . ”), the entire disclosure of which is hereby incorporated by reference into this specification. This patent claims: “1. A method of inhibiting a thioredoxin/thioredoxin reductase redox system in a cell comprised of contacting said cell with an effective amount of an agent that is an inhibitor of redox activity, said inhibitor having an IC50 TR/Trx of less than about 50 μg/nl and being selected from the group consisting of NSC 401005, NSC 208731, NSC 382000, NSC 665103, NSC 617145, NSC 618605, NSC 622378, NSC 620109, NSC 163027, NSC 131233, NSC 665102, NSC 631136, NSC 681277, NSC 140377, NSC 603084, NSC 382007, NSC 635002, NSC 620358, NSC 657028, NSC 661211, NSC 622188, NSC 645330, NSC 350629, NSC 102817, NSC 626162, NSC 655897, NSC 267461, NSC 627124, NSC 610187, NSC 624982, NSC 664951, NSC 277293, NSC 608972, NSC 634761, NSC 664271, NSC 665878 and NSC 625814.”

The disclosure of such U.S. Pat. No. 6,372,772 is also of interest. The patent discloses (at lines 19-27 of column 1) that “Cellular redox systems appear to be very important to normal cellular activity. Cells maintain an intracellular environment that is reducing in the face of a highly oxidizing extracellular environment. Regulated alterations in the intracellular redox state (redox signaling) can modulate events such as DNA synthesis, enzyme activation, selective gene expression, regulation of cell cycle, cell growth, and programmed cell death.”

U.S. Pat. No. 6,372,772 also discloses (at lines 28-33 of column 1) that “One of the more important consequence of intracellular redox signaling is a change in the oxidative state of select cysteine residues on certain proteins. The post-translational modification of cysteine is difficult to follow since it lacks a convenient marker and is readily reversed when the cell contents are exposed to extracellular oxidizing conditions.”

U.S. Pat. No. 6,372,772 also discloses (at lines 34-41 of column 1) that “One type of abnormal cell function is abnormal cellular proliferation. Abnormal cellular proliferation is a cardinal feature of human malignancy. During the past decade there has been much insight into the biomolecules that regulate cell proliferation and the pathways in which they operate. These biomolecules have been identified as pharmacological, therapeutic, and/or diagnostic targets for agents which inhibit cellular proliferation.”

U.S. Pat. No. 6,372,772 discusses resistance to apoptosis from lines 42 of column 1 to line 3 of column 2, stating that “Another type of abnormal cell function is resistance to apoptosis. Apoptosis is a form of programmed cell death characterized by membrane blebbing, chromatin margination and breakdown of chromosomal DNA into nucleosome-sized fragments. Programmed cell death or apoptosis is an important event in the normal processes of development and tissue remodeling. Loss of apoptosis can lead to diseases associated with cellular proliferation, such as cancer autoimmune disease, inflammation and hyperproliferation disease, while increased apoptosis can lead to neurodegenerative disease and destruction of tissue, as well as cardiovascular damage. Normally, when a cell sustains substantial genetic damage that cannot be repaired by normal DNA repair processes, this is recognized by sensory mechanisms in the cell and a sequence of events is initiated which leads to the death of the cell. Apoptosis results in the death of individual damaged cells and protects the organism from potentially harmful genetic changes that could lead to unregulated cell growth including cancer. Apoptosis resistance has been correlated with induction of the cyclin-dependent kinase inhibitor. There are now documented instances where inhibition of apoptosis by the abnormal expression of an oncogene or the loss of a tumor suppresser genes are closely associated with malignancy. It also appears that as cells develop from a nontransformed state, through a pre-malignant to a fully transformed state, they progressively lose their ability to undergo apoptosis. Apoptosis is also inhibited by some viral infections, in auto immune disease and hyperproliferative skin diseases.”

At columns 12 et seq. of U.S. Pat. No. 6,372,772, a process is described for determining agents that inhibit the thioredoxin system. It is disclosed (commencing at line 7 of column 12) that refers to “. . . an initial approach to developing agents which might selectively inhibit thioredoxin-dependent cell proliferation and/or apoptosis we studied a series of imidazolyl disulfides, typical of which is 1-methylpropyl 2-imidazolyl disulfide (IV-2). Prolonged incubation of thioredoxin with the alkyl 2-imidazolyl disulfide results in irreversible inhibition of the thioredoxin as a substrate for reduction by thioredoxin reductase. The inhibition appears to be specific for Cys 73 of thioredoxin, which remains a substrate for thioredoxin. In culture, IV-2 shows greater inhibition of thioredoxin than serum-dependent cell proliferation, suggesting that it may be producing its effect by inhibiting the thioredoxin redox system.”

United States patent also discloses (at lines 20-59 of column 12) that “Cell Screen: The compounds IV-2 and DLK-36 (benzyl-2-imidazolyl disulfide) another preferred disulfide are shown below . . . . IV-2 has also been shown to exhibit dose-dependent antitumor activity against human MCF-7 breast cancer and HL-60 xenografts growing in scid mice. IV-2 and the second disulfide, DLK-36, produced responses of seen 98% and 65% tumor inhibition respectively against the MCF-7 tumor system. IV-2 and DLK-36 showed antitumor activity against HL-60leukemia growing in scid mice with a number of the animals without tumor at day 45 for each compounds . . . The min (multiple intestinal neoplasia) mouse has been used herein, to test for chemopreventive activity of IV-2. The min mouse has a germline mutation in the APC gene seen in human familial adenomatous polyposis (FAP). This model was chosen because of its genetic basis and as an alternative to chemically-induced models of colon cancer. The majority of the tumors that develops in the adult min mice are in the small intestine, although tumors are first diagnosed in the colon. FAP represents a high risk group of subjects with a readily identifiable early end-point marker that is suitable for trials of novel chemopreventive agents. Compound IV-2 in the diet at 250 ppm (a dose less than optimal due to limited supply) reduced the number of tumors in the colon by 70% (p=0.0160) and caused a significant reduction in the size of remaining tumors (p<0.001 compared to control).”

Unite States patent also discloses (from line 60 of column 12 to line 15 of column 13” that “Beyond the therapeutic activity of thioredoxin reductase and thioredoxin and inhibitors thereof, new classes of inhibitors of both thioredoxin reductase and thioredoxin would be useful both as novel pharmacological probes for studying the roles of these enzymes in signal transduction pathways and as leads for structure optimization and structure/activity studies. Data from the National Cancer Institute's (NCI's) panel of 60 human cancer cell lines (as described by Monks et al., Feasibility of a high-flux anticancer drug screen using a diverse panel of cultured human tumor cell lines. Journal of the National Cancer Institute, 83, 757, (1991), which is hereby incorporated by reference for its teaching relating to screening) was analyzed using the COMPARE pattern recognition program (as described by Paull et al., Display and analysis of patterns of differential activity of drugs against human tumor cell lines: development of mean graph and COMPARE algorithm. Journal of the National Cancer Institute, 81, 1088, (1989), which is hereby incorporated by reference for its teaching relating to the COMPARE algorithm) with the two disulfide drugs (IV-2 and DLK-36) as seed compounds in order to identify other compounds with similar patterns of growth inhibition or as inhibitors of the thioredoxin redox system . . . . The preselection of molecules using the COMPARE analysis allow us to pursue molecular targets knowing in advance the growth inhibitory effects of the compounds and accordingly, reduces the number of compounds that must be tested from among over 50,000 compounds already screened in the NCI investigational drug data base. Using this cell directed screening approach (CDSA) we have identified a number of inhibitors of the thioredoxin redox system with a hit rate in the primary screen of 77% for compounds with IC50 s<=10 μg/ml compared to a hit rate of 3 to 10% by screening of non-related or randomly selected natural products.”

The materials and methods used in the process of U.S. Pat. Nos. 6,372,772 were described from line 27 of column 13 to line 4 of column 14, wherein it was disclosed that “The materials and methods of the present invention are as follows: Enzymes: Thioredoxin reductase, specific activity 43.6 μmole NADPH reduced/min/mg protein at 21° C., was purified from human placenta as previously described (Oblong et al., 1993) Glutathione reductase, specific activity 141.2 μmole NADPH reduced/min/mg protein at 21° C., was purified from aged human red blood cells (Colmon & Black, 1965). Human recombinant thioredoxin was expressed in E. coli and purified as previously described (Gasdaska et al., 1994). The thioredoxin was stored at −20° C. with 5 mM dithiothreitol which was removed before use with a desalting column (PD 10, Pharmacia, Uppsala, Sweden) . . . Assays: Microtitre plate colorimetric assays, based on the increase in absorbance at 405 nM which occurs as dithionitrobenzoic acid (DTNB) is reduced by the enzyme-mediated transfer of reducing equivalents from NADPH, were developed for thioredoxin reductase, thioredoxin reductase/thioredoxin-dependent insulin-reduction and glutathione reductase. Thioredoxin reductase/thioredoxin-dependent insulin reducing activity was measured in an incubation with a final volume of 60 μl containing 100 mM HEPES buffer, pH 7.2, 5 mM EDTA (HE buffer), 1 mM NADPH, 1.0 μM thioredoxin reductase, 0.8 μM thioredoxin and 2.5 mg/ml bovine insulin. Incubations were for 30 min at 37° C. in flat-bottom 96 well microtitre plates. The reaction was stopped by the addition of 100 μl 6 M guanidine HCl, 50 mM Tris pH 8.0, and 10 mM DTNB and the absorbance measured at 405 nM . . . . Assays of thioredoxin reductase or glutathione reductase were run in flat bottom 96-well microtitre plates thioredoxin reductase activity was measured in a final incubation volume of 60 μl containing HE buffer, 10 mM DTNB, 1.0 μM thioredoxin reductase and 1 mM NADPH. Glutathione reductase activity was measured in a similar assay in which thioredoxin reductase was replaced by 1.0 μM glutathione reductase. Compounds were diluted in HE buffer and added to the wells as a 20 μl aliquot, and thioredoxin reductase or glutathione reductase were then added also as a 20 μl aliquot in HE buffer. To ensure uniform coverage of the bottom of the well the plate was briefly spun at 3000×g. To start the reaction NADPH and DTNB were added as a 20 μl aliquot in HE buffer and the plate was moved to the plate reader preheated to 37° C. The optical density at 405 nm was measured every 10 sec and initial linear reaction rates measured.”

Several compounds were identified “. . . as inhibitors of thioredoxin reductase . . . ,” and they were described from lines 6 of column 14 through line 44 of column 17. In this section of the specification of U.S. Pat. No. 6,372,772, it was disclosed that “These compounds were identified as inhibitors of thioredoxin reductase and they were tested for cytotoxicity in the NCI's human cancer cell line panel of 60 human cancer lines (Monks et al., 1991). The IV-2 and DLK-36, which we have shown to be inhibitors of thioredoxin reductase with Ki values of 30.8 RM and 30.9 RM, respectively were used as seed compounds. The COMPARE pattern recognition program was also used to determine a Pearson correlation coefficient in order to rank the similarity of the pattern of growth inhibition caused by compounds from the NCI database of investigational drugs with the patterns of growth inhibition caused by IV-2 and DLK-36. Ninety-two compounds with similar patterns of activity from 50,000 compounds already tested in the cell screen were identified. Of the 92 compounds, 47 were non-discreet and available for further study. The correlation coefficients for the top 100 compounds lay between 0.836 and 0.718. Forty-seven of these compounds were non-discrete and available in sufficient quantities for further testing. As a negative control for the COMPARE evaluation process, an additional 52 non-discrete compounds were selected based on the lack of correlation between their patterns of cytotoxicity and that of DLK-36. A library of 221 randomly chosen natural products comprising a mixture of plant derived alkaloids and other compounds and pure bacterial products were also tested for their ability to inhibit thioredoxin reductase/thioredoxin-dependent insulin reduction. Stock solutions of the compounds, 10 mg/ml, were made in dimethylsulfoxide and stored at −20° C. Serial dilutions from these stock solutions were made in HE buffer immediately before use. The small amount of dimethylsulfoxide into the assays had no effect on activities. In the initial screen the agents were preincubated at room temperature for 30 minutes with thioredoxin reductase, glutathione reductase or thioredoxin reductase/thioredoxin in a volume of 40 μl after which remaining components of the assay were added as a 20 μl aliquot. The activities of some agents were also evaluated in subsequent assays without the preincubation . . . . Results: The linearity of all assays was verified with respect to time and to the amounts of glutathione reductase, thioredoxin reductase or thioredoxin. For the thioredoxin reductase/thioredoxin-dependent insulin reduction assay the variance for 12 control assays was ±0.05%. For assays of thioredoxin reductase and glutathione reductase the variance was ±0.18%. Using replicate assays the lower limit for reproducible detection of inhibition is about 5% inhibition.”

Table 1 of U.S. Pat. No. 6,372,772 (see columns 14 and 15) described the “. . . compounds having IC50<=50 μg/ml are shown below in Table I. Of 47 non-discrete compounds tested in a thioredoxin reductase/thioredoxin insulin reduction assay, 36 (77%) were inhibitors with IC50 s<=10 μg/ml and 15 of those (32%) had IC50 s<=1 μ/ml . . . . The range of Pearson correlation coefficients compared to the seed compounds IV-2 or DLK-36 for these compounds was 0.836 (NSC 401005) to 0.718 (NSC 603084). The concentration causing 50% inhibition of activity in the thioredoxin reductase/thioredoxin-dependent insulin reduction assay, ranked from the most to the least active compounds, is given. Also given is the mean 50% growth inhibition concentration (GI50) of the compounds for the entire cell line panel and for leukemias which was the most sensitive human tumor type . . . . The structures of the 8 most potent inhibitors IC50 TRTrx<=0.15 μg/ml) in the thioredoxin reductase/thioredoxin-dependent insulin reduction assay are shown below:” At columns 16-17, the eight most potent inhibitors of the thioredoxin system found by the patentees were shown. [Figure]

At columns 17 et seq. of U.S. Pat. No. 6,372,772, the patentees went on to disclose that “In comparison, a similar screening assay of 52 compounds from the NCI database whose activity was not related to that of the seed compound DLK-36 yielded only 5 compounds (10%) with IC50 s<=10 μg/ml and 1 compound (2%) with an IC50<=μg/ml. A screening assay of 221 randomly selected pure compounds derived from plant and microbial sources identified 6 compounds (3%) with IC50<=10 μg/ml and 3 compounds (1.5%) with IC50<=1 μg/l . . . . As secondary screens we used assays of thioredoxin reductase alone, to estimate the contribution of its inhibition to the inhibition seen in the primary screen, and glutathione reductase, to obtain estimates of the specificity of reductase inhibition. This is shown below in Table II:” This Table II was presented in the middle of column 17 of U.S. Pat. No. 6,372,772.

In such Table II, the concentrations of compound causing 50% inhibition of activity in the thioredoxin reductase/thioredoxin-dependent insulin reduction assay (IC50-TRTrx), in the thioredoxin reductase assay (IC50 TR) or in the glutathione reductase assay (IC50 GR) were ranked in decreasing order of potency of inhibition of the thioredoxin reductase/thioredoxin-dependent insulin assay. Also discussed in such Table II were the ratio of the IC50 in the thioredoxin-dependent assay to the IC50 in the assay of thioredoxin reductase alone (TR/TRTrx), and the ratio of the IC50 in the glutathione reductase assay to the IC50 in the thioredoxin reductase assay.

Commencing at line 58 of column 17, U.S. Pat. No. 6,372,772 discloses that “Of 24 compounds which were potent inhibitors of thioredoxin reductase/thioredoxin-dependent insulin reduction, 13 were at least 10-fold more selective as inhibitors of thioredoxin reductase compared to glutathione reductase with 6 of the compounds showing 100-fold selectivity. Of the most potent compounds, four show greater than 100 fold selectivity for the inhibition of thioredoxin reductase of versus glutathione reductase (NSC 401005, NSC 617145, NSC 618605 and NSC 622378). Additionally, the following compounds (NSC 620109 and 681277) show greater than 100 fold selectivity for the inhibition of thioredoxin reductase of versus glutathione reductase.” Two compounds, one of which was cyclic, were then shown in the first Figure of column 18.

U.S. Pat. No. 6,372,772 then disclosed several compounds that inhibited both thioredoxin and thioredoxin reductase. Commencing at line 19 of column 18 of U.S. Pat. No. 6,372,772, it was disclosed that “In addition, many of the compounds identified appeared to be specific for either thioredoxin reductase or thioredoxin (including its interaction with thioredoxin reductase) . . . . There were several compounds that, while active in the thioredoxin reductase/thioredoxin-dependent insulin assay, did not inhibit thioredoxin reductase alone suggesting that these compounds inhibit thioredoxin. These compounds include NSC 665103 (shown above), NSC 645330, and NSC 163027 (both of which are shown below) . . . In the NCI cell line panel the sensitivity of the leukemias to growth inhibition by the selected compounds was consistently greater than the mean sensitivity of all the cell lines (see Table II). A number of the compounds showed patterns of selective growth inhibition for colon, renal and breast cancer cell lines (data not shown).”

Commencing at line 52 of column 18 of U.S. Pat. No. 6,372,772, then patentees then discussed “. . . other structures . . . ” that inhibit thioredoxin/thioredoxin reductase. It is disclosed that “The compounds of the present invention may identify other structures (i.e. related compounds) which inhibit thioredoxin/thioredoxin reductase. Although not wishing to be bound by theory, a commonality appears exist among the structures of the compounds which inhibited thioredoxin reductase/thioredoxin-dependent insulin reduction. This commonality appears to be the alpha.-13-unsaturated keto moiety with highly electron-deficient centers. The high degree of halogenation of a number of these agents may also contribute to their reactivity. Apart from the ability of these electron deficient moieties to target the thiol functions of thioredoxin reductase and thioredoxin, the specificity of the agent for inhibition may arise from interaction of the multiple carbonyl functions with positively charged histidine, or e-amino residues of lysine.”

Formulations of the thioredoxin reductase inhibitors, and/or administration thereof, are discussed at columns 19 et seq. of U.S. Pat. No. 6,372,772. It is disclosed that “The method of this invention involves administering to a mammalian host, preferably a human host, pharmacologically effective amounts of one inhibitor of redox signaling. The inhibitors (i.e. the NSC compounds described above), may be combined in vitro before administration or separately administered to the host with other anticancer agents, in either order or concurrently or simultaneously, with administration generally taking place up to 24 hours after the administration of the other biological active agent(s).” It should be noted that, in the composition of the instant application, in addition to the thioredoxin system inhibitor, or in place thereof, one may use such “. . . inhibitor of redox activity . . . . ”

At lines 9 et seq. of U.S. Pat. No. 6,372,772, it is disclosed that “The adrninistration(s) may take place by any suitable technique, including oral, subcutaneous and parenteral administration, preferably parenteral or oral. Examples of parenteral administration include intravenous, intraarterial, intramuscular, and intraperitoneal, with intraperitoneal and intravenous being preferred. The dose and dosage regimen will depend mainly on whether the inhibitors are being administered for therapeutic or prophylactic purposes, separately or as a mixture, the type of biological damage and host, the history of the host, and the type of inhibitors or biologically active agent. The amount must be effective to achieve an enhanced therapeutic index as defined above. It is noted that humans are treated longer than the mice and rats with a length proportional to the length of the disease process and drug effectiveness. The doses may be single doses or multiple doses over a period of several days, but single doses are preferred. For purposes herein, a protection level of at least 50% means that at least 50% of the treated hosts exhibit improvement against the disease or infection, including but not limited to improved survival rate, more rapid recovery, or improvement or elimination of symptoms. The doses may be single doses or multiple doses. If multiple doses are employed, as preferred, the frequency of administration will depend, for example, on the type of host and type of cancer, dosage amounts, etc. For some types of cancers or cancer lines, daily administration may be effective, whereas for others, administration every other day or every third day may be effective, but daily administration ineffective. The practitioner will be able to ascertain upon routine experimentation which route of administration and frequency of administration are most effective in any particular case. The dosage amounts for cancer which appear to be most effective herein are those that result in regression in size of the tumor or complete disappearance or non-reappearance of the tumor, and are not toxic or are acceptably toxic to the host patient. Generally, such conditions as fever, chills and general malaise are considered acceptable. The optimum dose levels may also depend on sequence of administration, existing tumor burden, are the type of precursor.”

U.S. Pat. No. 6,372,772 also discloses “(commencing at line 50 of column 19) that “Compounds and agents of the present invention, in conjunction with a pharmaceutically acceptable carrier, may be used for any of the therapeutic effects, discussed above. Such compositions may be in the form of an agent in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones”

“This embodiment of the invention relates to the administration of a pharmaceutical composition (an inhibitor), in conjunction with a pharmaceutically acceptable carrier, for any of the therapeutic effects discussed above.”

“In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of. Remington's Pharmaceutical Sciences (Maack Publishing Co., Easton, Pa.) hereby incorporated herein by reference in its entirety.”

“Pharmaceutical compositions for oral administration can be formulated using pharmaceutically acceptable carriers well known in the art in dosages suitable for oral administration Such carriers enable the pharmaceutical compositions to be formulated as tablets, pills, dragees, capsules, liquids, gels, syrups, slurries, suspensions, and the like, for ingestion by the patient.”

“Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.”

“Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound. i.e., dosage.”

“Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.”

“Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.”

“For topical or nasal administration, penetrants appropriate to the particular barrier to be permeated are used in the formulation. Such penetrants are generally known in the art.”

“The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.”

“The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use.”

“After pharmaceutical compositions have been prepared, they can be placed in an appropriate container and labeled for treatment of an indicated condition, For administration of TR/Trx inhibitors, such labeling would include amount, frequency, and method of administration.”

“Pharmaceutical compositions suitable for use in the invention include compositions wherein the active ingredients are contained in an effective amount to achieve the intended purpose. The determination of an effective dose is well within the capability of those skilled in the art.”

“For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans.”

“The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.”

“Normal dosage amounts may vary from 0.1 to 100,000 micrograms, up to a total dose of about 1 g, depending upon the route of administration. Guidance as to particular dosages and methods of delivery is provided in the literature and herein as well as generally available to practitioners in the art.”

The formulation processes and compositions described in U.S. Pat. No. 6,372,772 may also be used with regard to some, all, or none of the components of applicant's composition.

At column 21 of U.S. Pat. No. 6,372,772, commencing at line 49, it is disclosed that “The NSC composition which inhibit thioredoxin and/or thioredoxin reductase and induces apoptosis, will enhance the antitumor activity of clinically used anticancer agents by contributing to tumor cell killing. This will enhance the antitumor activity of the clinical agent, especially for those agents where tumor cells have become resistant to the antitumor agent through their elevated levels of thioredoxin and/or the lack of ability to apoptose. Therefore, in another embodiment, the NSC compounds may be administered in combination which clinically available agents used to treat cancer. These may include, but are not limited to, cisplatin, doxorubicin, etoposide, taxol, taxotere, tamoxifen, IL-2, methotrexate, and 5-fluorouracil.” In applicant's invention, such “NSC composition” is preferably administered together with one or more anti-tubulin agents with specified degrees of specificity for the beta_{II}, beta_{III}, and/or beta_{V} isotypes of tubulin.

Commencing at line 62 of U.S. Pat. No. 6,372,772, it is disclosed that “Tissue with elevated levels of thioredoxin may lose the ability to eliminate damaged cells through the process of apoptosis and therefore may lead to the development of cancer . . . . Therefore, in one embodiment, the NSC composition which inhibit thioredoxin and induce apoptosis, may be administered on a regular basis to induce apoptosis for the prevention of cancer . . . . Based on the measured in vivo treatment doses of the disulfides (IV-2, DLK-36), Table III shows the expected preferred IP dose ranges.” Such Table III is presented in the middle of column 22 of U.S. Pat. No. 6,372,772.

As will be apparent from the foregoing discussion, one may utilize the teachings of U.S. Pat. No. 6,372,772 to identify one or more thioredoxin inhibitors for use in the composition of the instant invention. Alternatively, or additionally, one may also use other redox inhibitors in the composition of this invention.

In one embodiment, candidate drugs targeting specific tubulin isotypes are titrated in wells containing human breast cancer cells as well as matched normal breast epithelial cell controls. The novel tubulin-targeting molecules of this invention, when compared to taxol, are more effective against cancer cells and less toxic to normal cells.

In one embodiment, human breast carcinoma (neoplastic) cell lines MDA-231 and MDA-MB-435 are purchased from the American Type Culture Collection. As a control, the human non-neoplastic mammary epithelial cell line HMEC is obtained from Clonetics. Cells are treated, 24 hours after plating, with the test compounds in eight serial dilutions. After 3 days in culture, the number of proliferating cells are measured by the MTS assay (described in Mossman T [1983], “Rapid colorimetric assay for cellular growth and survival: application to proliferation and cytoxicity assays,” J. Immunol. Methods 65, 55-63; an article by V. B. Segu et al., 1998, “Use of a soluble tetrazolium compound to assay metabolic activation of intact beta cells,” Metabolism: Clinical & Experimental 47, 824-830; and an article by G. Malich et al., 1997, “The sensitivity and specificity of the MTS tetrazolium assay for detecting the in vitro cytotoxicity of 20 chemicals using human cell lines,”. Toxicology 124, 179-192.). For each drug, there is calculated an IC₅₀ value for growth inhibition. Preferred criteria for success include 1) reduction of the cytotoxicity of the drug towards normal cells in comparison to taxol by a factor of 2-fold or more (in order to substantially expand the therapeutic window) and 2) lowering the IC₅₀ for cancer cells relative to taxol.

As an inhibitor of the thioredoxin system, there is preferably used the illudin derivative irofulven, that has been found to selectively inhibit both thioredoxin reductase and thioredoxin (described in B. A. Woynarowska et al., 2004, “Irofulven binding and inactivation of purified and celllar redox-controlling proteins,”. Proc. Amer. Assoc. Cancer Res. 45, 348 (abstract only).

In one embodiment, candidate tubulin-interactive drugs are tested in vivo in the murine xenograft models using the tumor cells described above. Thereafter, there are performed challenges in live mice with human cancers, using 10 tumored mice for each concentration of the isotype-specific drugs as well as for taxol and the untreated control. Mice are weighed and their tumor size measured twice weekly. Tumor growth inhibition is calculated.

The experiments are then repeated in the presence of irofulven (2.5 mg/kg, already shown to be tolerable for mice (described in Hammond L A, Hilsenbeck S G, Eckhardt S G, Marty J, Mangold G, MacDonald J R, Rowinsky E K, Von Hoff D D & Weitman S (2000) Enhanced antutumour activity of 6-hydroxymethylacylfulvene in combination with topotecan or paclitaxel in the MV522 lung carcinoma xenograft model. Eur. J. Cancer 36, 2430-2436) to see if irofulven improves the performance of the drugs.

The Use of Selective Anti-Tubulin Agents

In one preferred embodiment of this invention, the composition of this invention is comprised of at least one agent that selectively targets a beta-tubulin isotype. In one aspect of this embodiment, such beta-tubulin isotype is preferentially expressed in organisms that suffer from certain maladies.

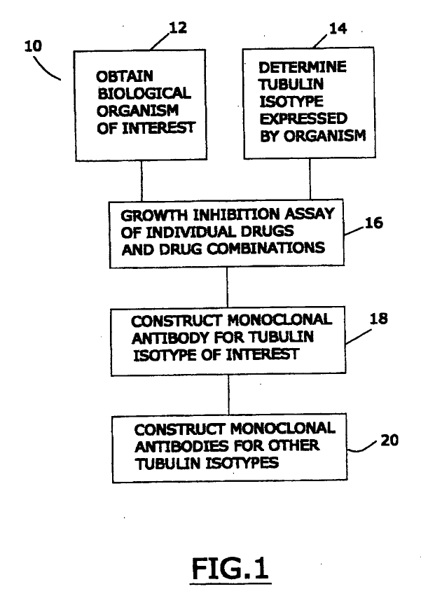

FIG. 1 is a flow diagram illustrating one preferred process 10 for identifying agents that selectively target certain beta tubulin isotypes. In step 12 of this preferred process, one preferably identifies a biological organism of interest by, e.g., obtain a stable transformed cell line.

The biological organism to be studied may, e.g., be one in which certain of the beta isotypes of tubulin are preferentially expressed when the organism is diseased. Several investigations have shown such a preferential expression of beta-tubulin in diseased organisms.

Thus, e.g., in an article by M. Haber et al. entitled “Altered expression of Mbeta2, the class II beta-tubulin isotype, in a murine J774.2 cell line with a high level of taxol resistance” (J. . Biol. Chem. 270, 31269-31275, 1995), it was reported that the levels of beta I and beta II rose 1.9-fold and 21-fold, respectively, in taxol-resistant murine cell lines (243).

Subsequently Ranganathan et al. published two articles on “Increase of beta III- and beta VIa tubulin isotypes in human prostate carcinoma cells as a result of estramustine resistance” (Cancer Res. 56, 2584-2589, 1996) and “Cloning and sequencing of human beta III-tubulin cDNA: induction of beta III isotype in human prostate carcinoma cells by acute exposure to antimicrotubule agents” (Biochim. Biophys. Acta 1395, 237-245, 1998). These articles reported that the levels of betaIII and betaIVa rose 4-9-fold and 3-5 fold, respectively, in estramustine-resistant DU-145 human prostate cancer cells.

Taxol-resistant MCF-7 human breast cancer cells were found to express increased levels of beta III, beta IVa and the tyrosinated form alpha-tubulin See, e.g., an article by 246. A. Banerjee on “Increased levels of tyrosinated alpha-, beta III, and beta IV tubulin isotypes in paclitaxel-resistant MCF-7 breast cancer cells” (Biochem. Biophys. Res. Commun. 293, 598-601, 2002).

It is well known that tumors expressing increased levels of beta III are more resistant to taxanes and estramustine. Reference may be had to articles by C. Dumontet et al. (2002, ““Expression of class III beta tubulin in non-small cell lung cancer is correlated with resistance to taxane chemotherapy,” Elect. J. Oncol. 1, 58-64.), by G. A. Orr et al. (2003, “Mechanisms of taxol resistance related to microtubules,”. Oncogene 22, 7280-7295), by A. Banerjee (2002, “Increased levels of tyrosinated alpha-, beta III, and beta_{IV}-tubulin isotypes in paclitaxel-resistant MCF-7 breast cancer cells,”. Biochem. Biophys. Res. Commun. 293, 598-601), by M. Kavallaris et al. (1997, “Taxol-resistant epithelial ovarian tumors are associated with altered expression of specific beta-tubulin isotypes,” J. Clin. Invest. 100, 1282-1293), by S. Sangrajang et al. (1998, “Association of estramustine resistance in human prostatic carcinoma cells with modified patterns of tubulin expression,”. Biochem. Pharmacol. 55, 325-331) and by P. Verdier-Pinard et al. (2003, “Analysis of tubulin isotypes and mutations from taxol-resistant cells by combined isoelectrofocusing and mass spectrometry,” Biochemistry 42, 5349-5357)

Almost as frequently observed is that increases in beta IV expression also accompany resistance to taxanes and vincristine. Reference may be had, e.g., to articles by F. M. Sirotnak et al. (2000, “Markedly decreased binding of vincristine to tubulin in Vinca alkaloid-resistant Chinese hamster cells is associated with selective overexpression of α and β tubulin isoforms,”. Biochem. Biophys. Res Commun. 269, 21-24), by A. N. Makarovsky et al. (2002, “Survival of docetaxel-resistant prostate cancer cells in vitro depends on phenotype alterations and continuity of drug exposure,” Cell. Mol. Life Sci. 59, 1198-1211), and by C. M. Galmarini et al. (2003, “Drug resistance associated with loss of p53 involves extensive alterations in microtubule composition and dynamics, Br. J. Cancer 88, 1793-1799).

In many fewer cases, taxane resistance involves increased expression of beta I (see an article by P. Giannakakou et al. [1997, “Paclitaxel-resistant human ovarian cancer cells have mutant beta-tubulins that exhibit impaired paclitaxel-driven polymerization. J. Biol. Chem. 272, 17118-17125]) or beta II. (see the aforementioned 2003 article by G. A. Orr relating to “Mechanisms of taxol resistance, the aforementioned 1995 article by M. Haber et al. on “Altered expression of Mbeta2, the class II beta-tubulin isotype, in a murine J774.2 cell line with a high level of taxol resistance,” and an article by C. Bernard-Marty et al. [2002, “Microtubule-associated parameters as predictive markers of docetaxel activity in advanced breast cancer patients: results of a pilot study,”. Clin. Breast Cancer 3, 341-345]).

Increased beta I expression also correlates with resistance to vincristine and E7010; see an article by F. M. Sirotnak et al. (2000, “Markedly decreased binding of vincristine to tubulin in Vinca alkaloid-resistant Chinese hamster cells is associated with selective overexpression of alpha and beta tubulin isoforms. Biochem. Biophys. Res. Commun. 269, 21-24.), and also to an article by Y. Iwamoto et al. (1998, “Preferential binding of E7010 to murine beta III-tubulin and decreased beta III-tubulin in E7010-resistant cell lines. Jpn. J. Cancer Res. 89, 954-962). Thus, there is ample evidence to suggest that cells alter the synthesis of certain tubulin isotypes in order to survive drug exposure. However, the mechanism by which the expression of specific tubulin isotypes is altered in drug-resistant cancer cells is still obscure.

Referring again to FIG. 1, and in the preferred embodiment depicted therein, the biological organism of interest may be one in which one or more of the tubulin isotypes have an amino acid sequence homology of less than about 90 percent with the other tubulin isotypes in the organism.

It is known that the differences in amino acid sequence among the isotypes of a given organism are generally clustered at the C-terminal ends. The fact that the sequences of the C-termini are usually highly conserved in evolution, even to minor differences, indicates that the C-termini are important. In addition, the C-termini contain the sites of most of the post-translational modifications, including phosphorylation, tyrosinolation/detyrosinolation, deglutamylation, polyglutamylation and polyglycylation.

The C-termini are highly negatively charged. Without wishing to be bound to any particular theory, applicant believes that the C-termini are likely to be projecting outward from the tubulin dimer and the microtubule. One might imagine that the C-terminus serves as a signal for other proteins that help to determine the function of that isotype.

A beta-tubulin, with the sequence EGEFEEE near its C-terminus, is likely to form an axoneme. Reference may be had, e.g., to an article by E. C. Raff et al. on “Microtubule architecture specified by a beta-tubulin isoform,” Science 275, 70-73. (36).

In an article by Fackenthal et al. (1993, “Tissue-specific microtubule functions in Drosophila spermatogenesis require the beta-tubulin isotype-specific carboxy-terminus,”. Dev. Biol. 158, 213-227), it was reported that removal of the C-terminus from the axonemal beta II isotype in Drosophila did not prevent that isotype from forming the axonemal microtubules, but those axonemes were not functional. Clearly, the signal sequence is necessary for successful function in the case of this isotype.

The C-termini of alpha- and beta-tubulin are also the sites where a variety of proteins bind; these include MAP2, tau, calponin and the motor protein Ncd Reference may be had, e.g., to articles by U. Z. Littauer et al. (1986, “Common and distinct tubulin binding sites for microtubule-associated proteins,” Proc. Nat. Acad. Sci. USA 83, 7162-7166), by T. Fujii et al. (1999, “Identification of the binding region of basic calponin on α and β tubulins,”. J. Biochem. 125, 869-875), by A. Karabay et al. (2003, “Identification of Ncd tail domain-binding sites on the tubulin dimer,”. Biochem. Biophys. Res. Commun. 305, 523-528), and by R. G. Burns et al. (1990, “Analysis of β-tubulin sequences reveals highly conserved, coordinated amino acid substitutions. Evidence that these ‘hot spots’ are directly involved in the conformational change required for dynamic instability,” FEBS Lett. 271, 1-8).

In the 1990 Burns et al. article, there was noticed a correlation between the nature of the aromatic amino acid near the C-terminus of beta isotypes and the amino acid at position 217/218. If the former is a tyrosine then the latter two are both threonines, while if the former is a phenylalanine, then the latter are other residues. Without wishing to be bound to any particular theory, applicant believes that the C-terminus may occasionally lie down along the microtubule and interact with the residues at position 217/218. Thus, the “visibility” of the signal sequence may vary depending on circumstances.

Applicant believes that the C-terminal sequence is not the whole story, however. Tubulin isotypes differ from each other at other places besides their C-termini. The lack of the assembly-critical cys239 in mammalian beta III is a case in point. Hoyle et al. (1995, “Regulation of beta-tubulin beta III function and expression in Drosophila spermatogenesis. Dev. Genet. 16, 148-170) described an experiment in which they prepared a chimera of Drosophila b2 in which positions 1-344 were replaced by the corresponding sequence of beta III. The remainder of the beta II contained the C-terminal sequence. Beta II is the axonemal and meiotic isbtype. If the C-terminal sequence were all that mattered then the chimeric tubulin should function equally well. In reality, the chimeric protein did not form outer doublet microtubules very well and was not able to carry out meiosis successfully. Thus, applicant believes that parts of the protein other than the C-termini must play a role in determining isotype function.

Other evidence supports applicant's hypothesis. For example applicant has observed a difference in the conformational rigidity among the alpha/beta II, alpha/beta III and alpha/beta IV dimers in the region in which we can artificially form a cross-link between cys12 and either cys201 or cys211 (Sharma, J., and Ludueña, R. F.[1994], “Use of N,N′-polymethylenebis(iodoacetamide) derivatives as probes for the detection of conformational differences in tubulin isotypes,” J. Prot. Chem. 13, 165-176).

Modeling studies indicate that this region is the binding pocket for the exchangeable GTP and that GTP binding is influenced by conformational changes in this region. See, e.g., an article by O. Keskin et al.(2002, “Relating molecular flexibility to function: a case study of tubulin,” Biophys. J. 83, 663-680).

It is believed that the kinetics of hydrolysis of this GTP, which determine the dynamic properties of the microtubule, will certainly be influenced by the conformational rigidity in this area, which in turn depends on the nature of the isotype. Similarly, the lateral and longitudinal bond energies in the microtubule have been estimated and could easily vary among the isotypes. Reference may be had, e.g., to an article by V. Van Buren (2002, “Estimates of lateral and longitudinal bond energies within the microtubule lattice,”. Proc. Nat. Acad. Sci. USA 99, 6035-6040).

Specific amino acid substitutions at positions involved in lateral tubulin/tubulin interactions have been shown to promote cold stability. See, e.g., articles by H. W. Detrich et al. (2000, “Cold adaptation of microtubule assembly and dynamics. Structural interpretation of primary sequence changes present in the alpha- and beta-tubulins of Antarctic fishes,” J. Biol. Chem. 275, 37038-37047), and by S. Pucciarelli et al. “(2002, “Characterization of the cold-adapted alpha-tubulin from the psychrophilic ciliate Euplotes focardii,” Extremophiles 6, 385-389).

Without wishing to be bound to any particular theory, applicant believes that the C-terminal sequence serves as a signal to other cellular proteins to determine at which cellular location, or in which population of microtubules, the isotype will perform its function. The rest of the protein is necessary for that function to be performed properly.

In one embodiment, and referring again to FIG. 1, in step 12 a stable transformed cell line is obtained. As is known to those skilled in the art, these stable transformed cell lines allow the study of individual proteins in isolation. Reference may be had, e.g., to U.S. Pat. Nos. 4,608,339, 5,804,431 (method, compositions, and apparatus for cell tranfection), 5,654,185, 5,811,274 (methods, compositions, and apparatus for cell transfection), 6,528,312, and the like. The entire disclosure of each of these United States patents is hereby incorporated by reference into this specification.

Referring again to the process depicted in FIG. 1, and in step 14 thereof, one can determine the tubulin isotypes expressed by the organism. This may be done by conventional means. Thus, e.g., one may isolate the primary cell-line culture in order to determine the tubulin isotypes expressed by the organism. Thereafter, in step 16 thereof, one can determine which of such tubulin isotypes are differentially expressed when certain conditions are present.

One may determine by reference to the literature which isotypes are expressed and/or differentially expressed by a particular organism.

Thus, for example, it is very likely that the genus Homo (human) expresses seven alpha isotypes, nine beta isotypes, and two gamma isotypes of tubulin. Reference may be had, e.g., to articles by L. T. Yeh and R. F. Luduena (2004, “The beta II isotype of tubulin is present in the cell nuclei of a variety of cancers,” Cell Motil. Cytoskeleton 57, 96-106), by C. DuMontet et al, (1999, “Expression of a new beta tubulin isotype in brain,” Mol Biol. Cell 10, 141a), by F. Stanchi et al. (2000, “TUBA8: a new tissue-specific isoform of alpha-tubulin that is highly conserved in human and mouse,” Biochem.Biophys. Res. Commun. 270, 1111-1118), by M. Litte et al. (1988, “Comparative analysis of tubulin sequences,” Comp. Biochem. Physiol. B 90B, 655-670), and by S. A. Lewis et al. (1985, “Three expressed sequences within the human beta-tubulin multigene family each define a distinct isotype,” J. Mol. Biol. 182, 11-20).

The “C-terminal sequences” for the various beta-tubulin isotypes found in human beings are also known. Information about the C-terminal sequences of tubulin isotypes is readily available in the literature. Reference may be had, e.g., an article by D. Wang et al. (1986, “The mammalian beta-tubulin repertoire: hematopoietic expression of a novel heterologous beta-tubulin isotype,” J. Cell Biol. 103, 1903-1910), by M. G. Lee et al. (1983, “Evolutionary history of a multigene family: an expressed human beta-tubulin gene and three processed pseudogenes,” Cell 33, 477-487), by D. V. Crabtree et al. (2001, “Tubulins in the primate retina: evidence that xanthophylls may be endogenous ligands for the paclitaxel-binding site,” Bioorg. Med. Chem. 9, 1967-1976). by C. DuMontet et al. (1999, “Expression of a new beta tubulin isotype in brain,” Mol. Biol. Cell 10, 141a), by M. Van Geel et al. (2002,) Identification of a novel β-tubulin subfamily with one member (TUBB4Q) located near the telomere of chromosome region 4q35,”. Cytogenet. Cell Genet. 88, 316-321, by S. Ranganathan et al. (1998, “Cloning and sequencing of human beta III-tubulin cDNA: induction of beta III isotype in human prostate carcinoma cells by acute exposure to antimicrotubule agents,” Biochim. Biophys. Acta 1395, 237-245), by K. Arai (2001, “Preparation and characterization of a monoclonal antibody to class II beta-tubulin isotype,”. NCBI Accession # BAB72260), by M. G. Lee et al. (1984, “Sequence of an expressed human beta-tubulin gene containing ten Alu family members,”. Nucleic Acids Res. 12, 5823-5836), by R. L. Strausberg et al. (2002, “Generation and initial analysis of more than 15,000 full-length human and mouse cDNA sequences,”. Proc. Nat. Acad. Sci. U.S.A. 99, 16899-16903), in Swissprot Accession #'s Q9BUF5 and Q8AVU1) and by J. Adachi et al. (2000, Swissprot Accession # Q9CUN8).

In one embodiment, and referring again to FIG. 1, the biological organism of interest is a bacterium. As is known to those skilled in the art, a bacterium is a minute, unicellular prokaryotic organism that is characterized as a lower protest; bacteria occur in soil, water, and air and as symbionts, parasites, or pathogens or humans and other animals, plants, and other microorganisms.

In one aspect of this embodiment, a drug is designed to target the FtsZ protein. FtsZ is a protein that is critically involved in bacterial cell division; and a drug targeting this protein would be very effective against bacteria. Inasmuch as animals, including humans, do not have any protein like FtsZ, the adverse side effects of such a specific drug would be minimal. Such a targeted drug would be useful for bacterial diseases that include, e.g., anthrax, leprosy, tuberculosis, plague, etc.

In another embodiment of the invention, the anti-tubulin drug is designed to be specific for a tubulin isotype that is present in one or more protists. As is known to those skilled in the art, a protist is a unicellular or multicellular organism that lacks the tissue differentiation and the elaborate organization that is characteristic of plants and animals; some protists are plant-like, some are animal-like, and some have properties common to both kingdoms. The taxon Protista includes algae and protozoa (all of which are eukaryotic protists).

Protists have tubulins that differ from human tubulin. Drugs that specifically bind to such tubulins would have little effect against human tubulin and, thus, would be useful against protist-caused diseases such as malaria, trypanosomiasis (sleeping sickness), Chagas disease, leishmaniasis; kala-azar; toxoplasmosis; babesiosis; cryptosporidiosis; trichomoniasis; giardiasis, emeric cocciciosis (disease of poultry) and amebic dysentery.

The isotypes of alpha-, beta-, and gamma-tubulin that are present in protists are well described in the literature. Reference may be had, e.g., to articles relating to Cryptosporidium (S. Caccio et al., 1997, “The beta-tubulin gene of Cryptosporidium parvum,” Mol. Biochem. Parasitol. 89, 4155-4159; R. G. Nelson et al., 1991, “Identification and isolation of Cryptosporidium parvum genes encoding microtubule and microfilament proteins,”. J. Protozool. 38, 52S-55S; and M. S. Abrahamsen et al., 2004, “Complete genome sequence of the apicomplexan, Cryptosporidium parvum. Science 304, 441-445), to Taxoplasma (S. D. Nagel et al., 1988, “The alpha- and beta-tubulins of Toxoplasma gondii are encoded by single copy genes containing multiple introns,” Mol. Biochem. Parasitol. 29, 261-273), to Babesia (R. E. Casu, 1993, “Sequence of a cDNA encoding beta-tubulin from Babesia bovis. Mol. Biochem. Parasitol. 59, 339-340), to Plasmodium (D. J. Rawlings et al., 1992, “beta-Tubulin II is a male-specific protein in Plasmodium falciparum,” Mol. Biochem. Parasitol. 56, 239-250; and S. Maessen et al., “The gamma-tubulin gene of the malaria parasite Plasmodium falciparum,” Mol. Biochem. Parasitol. 60, 27-35), and the like. The aforentioned listing of references is merely illustrative, and the tubulin isotypes of Eimeria, Physarum (slime mold), Chloromonas (snow alga), Chlamydomonas, Polytomella, Volvox, Paramecium, Tetrahymena, Stylonichia, Euplotes, Moneuplotes, Histriculus, Moneuplotes, Tintinnopsis, Strobilidium, Metacylis, Laboea, Strombidinopsis, Favella, Opisthonecta, Halteria, Metopus, Heliophrya, Nyctotherus, Dictostelium, Ectocarpus, Chondrus, Achlya, Amphidinium (dinoflagellate), Reticulomyxa, Naegleria, Leishmania, Trypanosoma, Trichomonas, Tritritchomonas, Trichonympha, Hypotrichomonas, Monocercomonas, Pelvetia (brown alga), Bigelowiella, Pyrsonympha, Streblomastix, and the like also may be readily determined from the literature.

In one preferred embodiment, and referring again to FIG. 1, the biological organism of interest is a plant. Plants have the protein tubulin that differs from human tubulin and also differs from plant to plant. One can determine the tubulin specific to undesired plants and, thereafter, design tubulin-isotype specific herbicides that would not kill or adversely affect humans, animals, or other plants.

The alpha, beta-, and gamma-isotypes of plants are well known to those skilled in the art. By way of illustration and not limitation, reference may be had, e.g., to articles relating to Pisum/pea (M.-F. Liaud et al., mann, H., and Cerff, R. (1992) The beta-tubulin gene family of pea: Primary structures, genomic organization and intron-dependent evolution of genes,” Plant Mol. Biol. 18, 639-651), to Solanum/potato (M. A. Taylor et al., 1994, “Characterization of the cDNA clones of two beta-tubulin genes and their expression in the potato plant . . . ,” Plant Mol. Biol. 26, 1013-1018), to Eucalyptus (E. C. Diaz et al, 1996, “Eucalypt alpha-tubulin: cDNA cloning and increased level of transcripts in ectomycorrhizal root system, Plant Mol. Biol. 31, 905-910), to Zinnia (T, Yoshimura et al., 1996, “Differential expression of three genes for different beta-tubulin isotypes during the initial culture of Zinnia mesophyll cells that divide and differentiate into tracheary elements,” Plant Cell Physiol. 37, 1167-1176), to Oryza/rice (D. Breviario et al., 1995, “Three rice cDNA clones encoding different beta-tubulin isotypes,” Plant Physiol. 108, 823-824; T, Koga-Ban et al., 1995, “cDNA sequences of three kinds of beta-tubulins from rice. DNA,” Res. 2, 21-26; X, Oin et al., 1997, “Molecular cloning of three rice alpha-tubulin isotypes: differential expression in tissues and during flower development,”. Biochim. Biophys. Acta 1354, 19-23; and Y. Kim et al., 2001, “Nucleotide sequence of a cDNA (OstubG2) encoding a gamma-tubulin in the rice plant (Oryza sativa),” NCBI Accession # 049068), to Triticum/wheat (G. Segal et al., 1996, NCBI Accession #'s AAD10487, AAD10488, AAD10489, AAD10490, AAD10492, AAD10493), to Nicotiana/tobacco (S. Okamura et al., 1999, “Isotype-specific changes in the amount of beta-tubulin RNA in synchronized tobacco BY2 cells,” Cell Struct. Funct. 24, 117-122; S. Okamura et al., 2000, NCBI Accession # AAB50565; S. Okamura et al., 2003, “β-Tubulin isotypes in the tobacco BY2 cell cycle, Cell Biol. Int. 27, 245-246; D. Breviario et al., 2001, “alpha-Tubulins from tobacco: gene cloning and expression studies,” NCBI Accession #'s CAD13176, CAD13177, CAD13178; and K. Jautz et al., 2000, “Characterization of gamma tubulin from tobacco,” NCBI Accession #'s CAC00547), to Hordeum/barley (J. Schroder, 2000, NCBI Accession #'s CAA10664CAA70891, CAB76916, CAB76380), to Gossypium/cotton (S. Ji et al., 2002, “Expression profile study and functional analysis of α- and β-tubulin isotypes during cotton fiber development,” NCBI Accession #'s AAL92026, AAN32988, AAN32989, AAN32991, AAN32995, AAQ92665, AAQ92664, AAQ92666, AAQ92667, and AAQ92668), to Lupinus/lupine (T. D. Vassilevskaia et al., 1996, “Developmental expression and regulation by light of two closely related β-tubulin genes in Lupinus albus,” Plant Mol. Biol. 32, 1185-1189; and N. J. M. Saibo et al., 2004, “Lupinus albus gamma-tubulin: mRNA and protein accumulation during development and in response to darkness, Planta 219, 201-211), to Populus/aspen (U. C. Kalluri et al., 2002, “Molecular cloning of tubulin cDNA from aspen xylem,” NCBI Accession # AA023139; and Y-S. Wang et al., 2003, “Isolation and characterization of cDNAs involved in vascular development of quaking aspen,” NCBI Accession #'s AA063773, AA063781); to Zea/corn (R. Villemur et al., 1994, “Characterization of four new beta-tubulin genes and their expression during male flower development in maize . . . ,” Plant Mol. Biol. 24, 295-315; H. J. Rogers et al., 1993, “Four members of the maize beta-tubulin gene family are expressed in the male gametophyte,” Plant J. 4, 875-882; J. Canaday et al., 1994, NCBI Accession # Q41808; and J. Canaday et al., 1995, “Identification of two maize cDNAs encoding gamma-tubulin,” NCBI Accession # CAA58670), to Elusine/goosegrass (E. Yamamoto et al., 1998, “alpha-Tubulin missense mutations correlate with antimicrotubule drug resistance in Eleusine indica,” Plant Cell 10, 297-308; and E. Yamamoto et al., 1999, “Molecular characterization of four beta-tubulin genes from dinitroaniline susceptible and resistant biotypes of Eleusine indica,”. Plant Mol. Biol. 39, 45-61), to Miscanthus (W. Wu et al., 2003, “Molecular cloning and evolutionary analysis of Miscanthus α-tubulin genes,” Am. J. Bot. 90, 1513-1521), to Anemia/fern (B. Moepps et al., 1993, “Characterization of the alpha and beta tubulin gene families from Anemia phyllitidis,”. NCBI Accession #'s CAA48929, CAA48930), to Physcomitrella/moss (T. Fujita et al., 2002, “Isolation of α tubulin cDNAs in Physcomitrella patens,”. NCBI Accession # BAC24799, BAC24800); A. Baur et al., 2003, “Six beta-tubulin genes from Physcomitrella patens,” NCBI Accession #'s AAQ88113, AAQ88114, AAQ88115, AAQ88116, AAQ88117, AAQ88118; and T. A. Wagner et al., 1999, “Characterization of gamma tubulin from Physcomitrella patens,”. NCBI Accession # AAD33883); and the like.

In one embodiment, and referring to FIG. 1, the biological organism of interest is a fungus. As is known to those skilled in the art, a fungus is a multicellular plant-like eukaryotic organism that is non-photosynthetic and that is devoid of chlorophyll; fungi generally contain a mycelium and are frequently coenocytic.

Fungi have tubulins that differ from human tubulin. Drugs that specifically bind to this fungal tubulin will be able to kill the fungus without any adverse side effects on a human patient, or in the case of fungal diseases that attack plants, the diseased host plant. Thus, these drugs will be able to treat fungal diseases such as colletotrichum (plant blight), pneumocystis pneumonia (commonly found in AIDS patients), septoria leaf spots (which attack tomatoes), histoplasmosis, candidiasis, aspergillosis, and the like.

The tubulin isotypes in fungi are well known. Reference may be had, e.g., to articles relating to Histoplasma (G. S. Harris et al., 1989, “Characterization of alpha and beta tubulin genes in the dimorphic fungus Histoplasma capsulatum,”. J. Gen. Microbiol. 135, 1817-1832), to Colletotrichum (D. G. Pannaccione et al, 1990, “Characterization of two divergent beta-tubulin genes from Colletotrichum graminicola,” Gene 86, 163-170; and Y. Takano et al., 2001 “Microtubule dynamics during infection-related morphogenesis of Colletotrichum lagenarium,”. Fungal Genet. Biol. 34, 107-121), to Candida (H. A. Smith et al, 1988, “Isolation and characterization of a β-tubulin gene from Candida albicans,” Gene 63, 53-63; S. Daly et al., 1997, “Isolation and characterization of a gene encoding alpha-tubulin from Candida albicans,”. Gene 187, 151-158; and B. Dujon et al., 2004, “Genome evolution in yeasts,”. Nature 430, 35-44), to Neurospora (M. J. Orbach et al., 1986, “Cloning and characterization of the gene for beta-tubulin from a benomyl-resistant mutant of Neurospora crassa and its use as a dominant selectable marker,” Mol. Cell. Biol. 6, 2452-2461; and S. Heckmann et al., “Primary structure of Neurospora crassa gamma-tubulin, Gene 199, 303-309), to Trichoderma (G. H. Goldman et al., 1993, “A nucleotide substitution in one of the beta-tubulin genes of Trichoderma viride confers resistance to the antimitotic drug methyl benzimidazole-2-yl-carbamate,”. Mol. Gen. Genet. 240, 73-80; and M. Mukherjee et al., 2003, “Homologous expression of a mutated beta-tubulin gene does not confer benomyl resistance on Trichoderma virens,”. J. Appl. Microbiol. 95, 861-867), to Botryotinia (S. Y. Park et al., 1997, “Isolation and characterization of a benomyl-resistant form of beta-tubulin-encoding gene from the phytopathogenic fungus Botryotinia fuckeliana,”. Molecules & Cells 7, 104-109), to Erysiphe/grass mildew (J. E. Sherwood et al., 1990, “Sequence of the Erysiphe graminis f. Sp. hordei gene encoding beta-tubulin,” Nucleic Acids Res. 18, 1052), to Epichloe (.A. D. Byrd et al., 1990, “The beta-tubulin gene of Epichloe typhina from perennial ryegrass (Lolium perenne),” Curr. Genet. 18, 347-354). to Saccharomyces (S. G. Sobel et al., 1995, “A highly divergent gamma-tubulin gene is essential for cell growth and proper microtubule organization in Saccharomyces cerevisiae,” J. Cell Biol. 131, 1775-1788; and Y. Hiraoka et al., 1984, “The NDA3 gene of fission yeast encodes β-tubulin: a cold sensitive nda3 mutation reversibly blocks spindle formation and chromosome movement in mitosis,”. Cell 39, 349-358), to Schizosaccharomyces (T. Toda et al., 1984, “Identification of the pleiotropic cell division cycle gene NDA2 as one of two different α-tubulin genes in Schizosaccharomyces pombe,” Cell 37, 233-242), to Pneumocystis (M. Dyer et al., 1992, “Cloning and sequence of a beta-tubulin cDNA from Pneumocystis carinii: Possible implications for drug therapy,” Mol. Microbiol. 6, 991-1001; and J. Zhang et al., 1993, “Cloning and characterization of an alpha-tubulin-encoding gene from rat-derived Pneumocystis carinii,”. Gene 123, 137-141), to Geotrichum (S. E. Gold et al., 1991, “Characterization of two beta-tubulin genes from Geotrichum candidum. Mol. Gen. Genet. 230, 104-112), to Conidiobolus, Rhizopus, Basidiobolus, Spiromyces, Mortierella, Powellomyces, Allomyces, Harpochytrium, and Glomus (P. J. Keeling et al., 2000, “Evidence from beta-tubulin phylogeny that microsporidia evolved from within the fungi,”. Mol. Biol. Evol. 17, 23-31; P. J. Keeling et al., 2003, “Congruent evidence from alpha-tubulin and beta-tubulin gene phylogenies for a zygomycete origin of microsporidia,”. Fungal Genet. Biol. 38, 298-309; and K. Voigt et al., 2003, “Oligonucleotide primers for the universal amplification of beta-tubulin genes facilitate phylogenetic analyses in the regnum Fungi. Org. Divers. Evol. 3, 185-194), to Suillus (J. T. Juuti et al., 2004, “Two phylogenetically highly distinct beta-tubulin genes of the basidiomycete Suillus bovines, NCBI Accession #'s CAG27308, CAG27309), to Cryptococcus (M. C. Cruz et al., 1997, “beta-Tubulin genes and the basis for benzimidazole sensitivity of the opportunistic fungus Cryptococcus neoformans,” Microbiology 143, 2003-2008), to Encephalitozoon (J. Li et al., 1996, “Tubulin genes from AIDS-associated microsporidia and implications for phylogeny and benzimidazole sensitivity,” Mol. Biochem. Parasitol. 78, 289-214; L. M. MacDonald et al., 2003, “Characterization of factors favoring the expression of soluble protozoan tubulin proteins in Escherichia coli. Protein,” Expr. Purif. 29, 117-122; and M. D. Katinka et al., 2001, “Genome sequence and gene compaction of the eukaryote parasite Encephalitozoon cuniculi,”. Nature 414, 450-453).

Referring again to FIG. 1, and in one preferred embodiment, the composition of this invention is directed against the tubulin in insects. The tubulin in insects is different from human tubulin and also varies from insect to insect. Anti-tubulin insecticides that bind to the tubulin of specific insects will be able to kill the specified inspect pest without any side effects on humans, animals, plants, or beneficial insects.

Referring to FIG. 1, and in one preferred embodiment thereof, at least one component of the composition of this invention is specifically designed to bind to the beta-III isotype of tubulin. The beta-III isotype of tubulin is found in many cancers, and its normal distribution is even more limited than is beta-II. A drug specific for beta-III should have little adverse effect upon organs and tissues that do not contain substantial amounts of beta-III.

In another embodiment, the drug is specifically designed to bind to the beta-V isotype of tubulin. The beta-V isotype of tubulin appears to be found in more cancers than is beta-III, and its normal distribution appears to be more limited than that of beta-III.

Referring again to FIG. 1, and to the preferred embodiment depicted therein, the tubulin isotype of interest is one that has less than about 90 percent of sequence identity with other isotypes in the organism.

One oddity of tubulin distribution is that, when an organism with multiple isotypes has an alpha or beta isotype of unusual sequence, that isotype is often associated with the reproductive system; and, because of such unusual sequence,drugs can be designed to selectively target the reproductive system of the organism.

In Drosophila, the alpha 4 isotype is only 67 percent identical to the other three alpha isotypes; and it is uniquely expressed in the oocyte and the early embroyer. See, e.g., an article by K. A. Matthews et al., “A functionally specialized alpha-tubulin is required for oocyte meiosis and cleavage mitosis in Drosophila,” Development 117, 977-991, 1993.

The mouse alphaTTT isotype shares only about 70 percent of the amino acid sequence identity with the other alpha isotypes, and it is expressed only in the testis of the mouse; see, e.g., an article by N. B. Hecht et al., “Localization of a highly divergent mammalian testicular alpha tubulin that is not detectable in brain,” Mol. Cell. Biol. 8, 996-1000, 1988.

Xeonpus has an unusual alpha isotype expressed in the ovary. See an article by W.-L. Wu et al., “Ovary-specific expression of a gene encoding a divergent alpha-tubulin isotype in Xenopus” Differentiation 58, 9-18, 1994.

The platyhelminth Schmidtea has a highly divergent alpha expressed only in the testis. See an article by F. Simoncelli et al., “Molecular characterization and expression of a divergent alpha-tubulin in planarium Schmidtea polychroa,” Biochim. Biophys. Acta 1629, 26-33, 2003.

Sunflower pollen has a unqiue alpha tubulin, much more basic than other alphas. It is believed to have a different tertiary structure with an altered H1/B2 loop facing into the interior of the microtubule. See an article by J. L. Evrard et al., “A Novel pollen-specific alpha-tubulin in sunflower: structure and characterization,” Plant Mol. Biol. 49, 611-620, 2002.

The fungus Colletotrichum has a divergent beta tubuln expressed only in its conidia. See an article by T. L. Burr et al., “Isolation, characterization, and expression of a second beta-tubulin-encoding gene from Colletotrichum gloeosporiodes f. Sp. aeschynomene,” Appl. Environ. Microbiol. 60, 4155-4159, 1994.

The protist Naegleria expresses three alpha isotypes, one of which is only 61.9 percent identical to the other two alpha isotypes. The unusual alpha isotype is not expressed in the flagellate but only in the dividing ameba, where it is found in the spindle. Reference may be had, e.g., to an article by S. Chung et al., “Cloning and characterization of a divergent alpha-tubulin that is expressed specifically in dividing amebae of Naegleria grubert,” Gene 293, 77-86, 2002.

It is not clear why these divergent isotypes are restricted to the reproductive tissues. In fact, not all of the highly divergent isotypes occur in reproductive tissues. Mammals and birds have a very divergent beta isotypewhose expression is restricted to haematopoietic tissues, including erythrocytes and platelets. See, e.g., articles by D. Wang et al. (“The mammalian beta-tubulin repertoire: hematopoietic expression of a novel heterologous beta-tubulin isotype,” J. Cell. Biol. 103, 1903-1910, 1986) and D. B. Murphy et al. (“The sequence and expression of the divergent beta-tubulin in chicken erythrocytes,” J. Biol. Chem. 262, 14305-14312, 1987).

The soybean produces a divergent beta isotype; low levels of this isotype are expressed in the cotyledon, and high levels in the hypocotyls, when the soybean is grown in the absence of light; see, e.g., an article by M. J. Guiltinan et al., “The isolation, characterization and sequence of two divergent beta-tubulin genes from soybean . . . ,” Plant Mol. Biol. 10, 171-184, 1987. Without wishing to be bound to any particular theory, applicant believes that it is possible that an isotype that performs only a single function is more likely to diverge in the course of evolution than one that is involved in a large number of processes.

Referring again to FIG. 1, and to the preferred embodiment depicted therein, in step 16 of the process 10 a growth inhibition assay is performed with the tubulin isotype of interest and with various candidate drugs.In one embodiment, one may use the well known growth inhibition assay to determine the extent to which a particular anti-tubulin drug is effective. This assay is described, e.g., in U.S. Pat. Nos. 5,147,652 (Autobiotics and their use in eliminating nonself cells in vivo), 5,248,66 (Methods for inhibiting neoplastic cell proliferation sing platelet factor 4), 6,096,724 (Pyrimidine derivatives and guanine derivatives, and their use in treating tumor cells), 6,207,146 (Gene expression in mammalian cells), 6,800,483 (compositions and methods for sensitizing and inhibiting growth of human tumor cells), and the like. The entire disclosure of each of these United States patent is hereby incorporated by reference into this specification.

By way of yet further illustration, and by reference to U.S. Pat. No. 5,874,536: (Proteins with Oncostatin M activity and process for their preparation), section 6.1.2 of the patent discloses that “Growth inhibitory activity (GIA) was measured by a dye binding assay. A375 melanoma cells (3-4×103) were seeded in a volume of 0.1 ml of DMEM containing 10% fetal bovine serum (FBS) in 96 well microtiter plates. Various concentrations of Oncostatin M were added in a volume of 0.1 ml, and incubation at 37° C. was continued for 72 hr. The culture medium was removed, cells were stained with crystal violet, and relative cell proliferation was quantitated by measuring bound dye on a microtiter plate reader (Genetic Systems Corp., Seattle, Wash.) by absorbance at 590 nm. Cellular proliferation in the presence of Oncostatin M was compared with proliferation in untreated samples, and is expressed as a percentage of inhibition of maximal growth. Samples were assayed in duplicate or triplicate. GIA units of Oncostatin M were determined from inhibition curves and are defined as the amount of protein neeeded to inhibit by 50% the growth of A375 cells in a standard assay. When GIA units were normalized for protein concentration, the coefficients of variation for the normalized values were generally <20%.”

Referring again to FIG. 1, and in step 16 thereof, the growth inhibition utilized therein may be of both individual drugs, and also of combinations of drugs.

Construction of Monoclonal Antibiodies for the Tubulin Isotypes of Interest

Referring again to FIG. 1, and to the preferred embodiment depicted therein, in step 18 of the process monoclonal antibodies are constructed that are specific for the particular tubulin isotype(s) of interest.

One may constuct the monoclonal antibody for the tubulin isotype of interest by means well known to those skilled in the art. Thus, by way of illustration and not limitation, it is known that mononclonal antibodies have been constructed for the mammalian betaI, betaII, betaIII, betaIV, and betaV isotypes. Reference may be had, e.g., to articles by M. C. Roach et al. (“Preparation of a monoclonal antibody specific for the class I isotype of beta-tubulin. The beta isotypes of tubulin differ in their cellular distributions within human tissues,” Cell. Motil. Cytoskeleton 39, 273-285, 1998), A. Banerjee et al. (“A monoclonal antibody against the type II isotype of beta-tubulin. Preparation of isotypically altered tublin,” J. Biol. Chem. 263, 3029-3034, 1988), by A. Banerjee et al. (“Preparation of a monoclonal antibody specific for the class IV isotype of beta-tubulin . . . ,” J. Biol. Chem. 267, 5625-5630, 1992), by A. Banerjee et al. (“Increased microtubule assembly in bovine bain tubulin lacking the type III isotype of beta tubulin, J. Biol. Chem. 265, 1794-1799, 1990), by M. K. Lee et al. (“The expression and posttranslational modification of neuron-specific beta-tubulin isotype during chick embryogenesis,” Cell Motil. Cytoskeleton 17, 118-132, 1990), by S. Lobert et al. (“Binding of vinblastine to phosphocellulose-purified and alphabeta-class III tubulin: the role of nucleotides and beta-tubulin isotypes,” Biochemistry 34, 8050-8060, 1995), and by S. Lobert et al. (“Energetics of Vinca alkaloid interactions with tubulin isotypes: implications for drug efficacy and toxicity,” Cell Motil. Cytoskeleton 39, 107-121, 1998).

By way of illustration and not limitation, excerpts from the “EXPERIMENTAL PROCEDURES” described at pages 5625-5626 of the 1992 A. Banerjee et al. paper will be set forth below.

“Materials—Peptides whose sequences correspond to the C-terminal portions of beta tubulin isotypes were synthesized by the BioSearch Corp. of San Rafael, Calif. The specific peptides were as follows: beta I-peptide, Cys-Glu-Glu-Ala-Glu-Glu-Glu-Ala-OH, corresponding to the C terminus of the beta I; beta II-peptide, Cys-Glu-Gly-Glu-Glu-Asp-Glu-Ala-OH, corresponding to the C terminus of chicken and pig β_{II}; β_{III}-peptide, Cys-Glu-Ser-Glu-Ser-Glu-Ser-Gln-Gly-Pro-Lys-OH, corresponding to the C-terminal sequence of human beta III; beta V-peptide, Cys-Glu-Ala-Glu-Glu-Glu-Val-Ala-OH, corresponding to the C-terminal sequence of rat, mouse, and human beta IV; and beta V-peptide, Cys-Glu-Glu-Glu-Ilu-Asn-Glu-OH, corresponding to the C-terminal sequence of chicken beta V. All of these peptides were used in the selection of a monoclonal antibody against the beta IV isotype. The beta II-peptide and the beta III-peptide were previously used to prepare the monoclonal antibodies JDR.3B8, here referred to as anti-beta II, and SDL.3D10, here referred to as anti-beta III, specific respectively for the beta II and beta III isotypes (Banerjee et al, 1998, 1990). GammaBind®-Plus was obtained from the Genex Corp. (Gaithersburg, Md.). Rign-A-[4-³H] colchicines was obtained from Amersham (United Kingdom). ¹²⁵I-labeled goat anti-mouse antibody was from ICN Biomedicals, Inc. (Costa Mesa, Calif.). Other materials were as described previously (Luduena et al., 1982). Samples of chimeric proteins, each consisting of the N terminus of Escherichia coli tryptophan synthetase E and the C-terminal ˜100 amino acids of either beta I, beta II, beta III, or beta IV was the kind gift of Dr. D. Cleveland, the John Hopkiins University, Baltimore, as was a polyclonal anti-beta I anti-serum”

As was also disclosed on page 5625 of the Banerjee et al. article, “Preparation of Tubulin Species—Microtubule protein was purified from bovine cerebra by cycling, and tubulin was purified there from by chromatography on phosphocellulose, according to the procedures of Fellous et al. (1977). This tubulin, to be referred to as PC tubulin, was the starting material for all further tubulin purifications. PC tubulin was further purified by immunoaffinity chromatography in order to study the properties of isotypically pure tubulin and was also fractionated to obtain tubulin to use in selecting the hydbridoma clone that produced antibody against beta IV. The use of immunaffinity chromatography to purify assembly-capable tubulin dimers with the compositions αβ_{II}, αβ_{III}, and αβ_{IV} will be described under Results.’”

“For the selection of the hybridoma clone, the following protein fractions were used.”

“1) α. PC-tubulin was reduced and carboxymethylated and subjected to preparative electrophoresis (Laemmli, 1970) on gels containing 5.5% polyacrylamide. The bands were visualized by treatment with 4M sodium acetate and were then cut out and eluted from the gel (Higgins and Dahmus, 1979). The protein form the a band, designated simply as α, consisted of all those forms of alpha-tubulin that form dimers with betaI, betaII, betaIII, and betaIV”

“2) AlphaIII,IV.PC-tubulin was chromatographed on an immunoaffinity column containing anti-betaIV. The column binds to all tubulin dimers containing beta I and beta II but does not bind to those which contain betaIII and betaIV (Banerjee et al., 1988). The tubulin that did not bind to the column was reduced and carboxymethylated and subjected to electrophoresis as above; the alpha band was cut and eluted. The resulting alpha is designated alphaIII,IV to indicate that it consists of those species of alpha that bind to beta_{III}, and β_{IV}.”

“3) Alpha I,II. The tubulin that bound to the anti- betaII column was eluted with 3 M KI and then reduced and carobyxmethylated and subjected to preparative electrophoresis, and the α band was purified as above. The resulting α is designated alphaI,II to indicate that it consist of those species of a that binds to betaI, and betaII.”

“4) BetaI, II. We have previously shown that the anti-betaII column will quantitatively remove from a tubulin sample all of the dimers containing betaI, and betaIi. The tubulin that bound to the anti-betaII column was eluted, reduced, carboxymethylated, and subjected to electrophoresis on preparative polyacrylamide gels. The β band was cut out and eluted. The resulting beta is designated betaI,II to indicate that it is a mixture of betaI and betaII.”

“5) Alpha/betaIII,IV. This is the tubulin that did not bind to the anti-betaII column.”

“6) BetaIII. The Alpha/betaIII,IV was reduced and carboxymethylated and subjected to preparative polyacrylamide gel electrophoresis as above. There were two beta bands on the gel. The slower one consisted only of betaIII; the faster moving one consisted only of betaIV (betaI and betaII also migrate in this position but were removed by passage through the anti-betaII column). The slower moving band was cut out and eluted from the gel and designated betaIII.”

“7) BetaIV . . . The faster moving band in the tubulin sample above, consisting entirely of betaIV, was cut out of the gel, and the tubulin eluted therefrom was designated betaIV.”

“Preparation of Microtubule-associated Proteins-Microtubules were purified from bovine cerebra as above, and . . . MAP 2 were purified by the procedure of Fellous et al. (1977).”

“Preparation of Monoclonal Antibody against βIV-tubulin. The βIV-peptide was conjugated with bovine serum albumin and m-maleimidobenzoyl-N-hydroxysuccinimide and injected into BALB/c mice. The selection of the mouse and preparation of hybridoma cell lines followed procedures that have previously been described (Banerjee et al., 1988). The fusion (with NS 1 mouse myeloma cells), initial screening, and determination of the resulting antibodies were performed by the Institutional Hybridoma Facility, Department of Microbiology, at the University of Texas Health Science Center at San Antonio.”

“The clones were selected as follows. Out of 1536 wells from the fusion, 47 tested positive by enzyme-linked immunosorbent assay against PC-tubulin and alpha/betaIII,IV. These 47 were further tested using betaI,II, betaIVe, betaV-peptide, alpha, betaIIIe , alphaIII, IV, and alphaI, II. Eleven wells were found whose contents tested strongly positive with βIVe and βV-peptide, slightly positive with βI,II, and negative to the others. Cells from these wells were subcloned against UV-irradiated thymocytes. After subcloning, 430 of the 1056 wells plated showed growth. Of these 430 wells, 132 tested positive against αβIII,IV. Eighteen monoclonal wells from these 132 were tested against beta I-peptide, beta II-peptide, beta III-peptide, beta IV-peptide, beta V-peptide, beta IVe, betaIVe, alpha, betaIIIe, and betaIVe. In addition, five mult-clonal wells from these 132 were subcloned into 960 wells, of which 30 tested positivre against alpha/beta III,IV. From these two groups of cell lines, 21 cell lines were found that tested positive against beta IVe and beta V-peptide and negative against alpha and the other beta isotypes. Fourteen of these clones were selected for further analysis. All were found to be IgG₁ (in contrast to anti-beta II and anti-beta III which were found to be IgG_{2B}). Of these, the nine which appeared most productive were tested for binding to protein A-agarose by an immunoprecipitation test. None of these appeared to bind to protein A-agarose by this test. Nevertheless, one of these clones, designated ONS.1A6, was chosen to be grown up in roller bottles. The antibody was purified from the supernatant on either a protein A-agarose or a GammaBind®-Plus column.”

Referring again to FIG. 1, and to step 20 thereof, monoclonal antibodies to other tubulin isotypes, such as the tubulin beta V isotype, can also be constructed in a similar manner. Reference may be had, e.g., to a 2003 publication by A. Banerjee et al. regarding the “Distribution and characterization of the beta V isotype of tubulin in mammalian cells,” Mol. Biol. Cell. 14, 182a. In one aspect of this embodiment, a C-terminal peptide specific for beta V tubulin CEEINE is synthesized, and the peptide is coupled to BSA or KLH using a conjugation kit obtained from Pierce, Inc. BSA-peptide and KLH-peptide conjugates are then dialyzed in sterile PBS and then stored frozen at 1 milligram/milliliter. Immunizations are then performed with female balb/C mice, which are immunized subcutaneously with 50 micrograms peptide-KLH in Complete Freund's adjvant. Three weeks later the mice are immunized again. After 500 days, 100 microliter samples are collected from each mouse by tail bleeding. The antibody titre of the sera is then tested by ELISA in a 96-well plate using BSA-peptide. To check the titre at different solutions, the sera are diluted in 5% non-fat dry milk in PBS.

The mice are thereafter sacrificed. The mouse's spleen is removed, and the spleen cells are collected. The spleen cells are fused with NS1 mouse myeloma cells in the presence of PEG-1500. The cells after fusion are diluted in HAT selection medium and are plated in 15 96-well plates and incubated in a carbon dioxide incubator for 10 days. After 10 days the plates are visually checked form the presence of colonies. The media from each colony are tested for the antibody titre in ELISA using β_{V} peptide-BAA.

Isolation of Microtubules

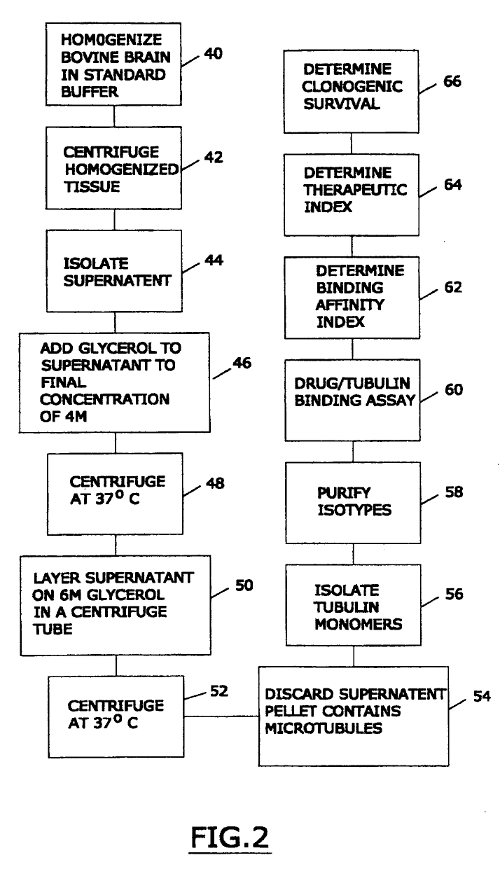

In FIG. 1, a process of identifying which candidate drugs selectively bind in vivo to certain tubulin isotypes was discussed. In FIG. 2, a process for isolating microtubules which can then be processed in accordance with this invention is discussed.

Referring to FIG. 2, and in step 40 thereof, mammalian tissue is homogenized in a standard phosphate buffer. One may use standard devices and processes for homogenizing such tissue. Reference may be had, e.g., to U.S. Pat. Nos. 3,750,964 (isolation and fractionation of organs of small animals), 4,307,864 (continuous flow tissue homogenizer), and the like. The entire disclosure of each of these United States patents is hereby incorporated by reference into this specification.

In one embodiment, and referring again to step 40 of FIG. 2, the mammalian tissue used is bovine brain tissue obtained from a slaughterhouse, and the standard phosphate buffer is “buffer A”. In one embodiment, it is preferred to remove the meninges from the tissue with tweezers before disposing the tissue in buffer and homogenizing it.

The mammalian tissue is preferably homogenized by first mixing the tissue with buffer A, at 4 degrees centigrade. Without wishing to be limited to a single buffer or set of conditions, one buffer that can be used for this protocol, the aforementioned “buffer A,” is formulated as follows: 100 millimolar 2-(N-morpholino)ethanesulfonic acid, pH 6.4, containing 0.1 mM ethylenediamine tetraacetic acid; 1 millimolar M ethylene glycol bis (β-aminoethyl ether) N,N,N′,N′-tetraacetic acid, 1 mM GTP, 0.5 mM MgCl₂, and 1 millimolar beta-mercaptoethanol. It may also optionally include glycerol to a final concentration of 4 molar; glycerol is often present during the purification procedure when one wants to make microtubules form), or 6 M in glycerol (during the purification procedure when it is desirable to centrifuge the microtubules through a layer of concentrated glycerol-containing buffer in order to remove them from non-microtubule proteins).

Referring again to FIG. 2, in step 42 of the process, the homogenzied tissue from step 40 is centrifuged, preferably at 4 degrees Celsius, until a solid and a liquid phase has been produced. the use of this temperature during centrifugation aids in separation of the supernatant liquid from the solid pellet and tends to prevent microtubule disassembly. Thereafter,the solid phase (the pellet) is discarded in step 44, thereby isolating the supernatant liquid.

The supernatent liquid thus isolated in step 44 is then preferably mixed with glycerol to a final concentration of 4 molar in step 46.

In step 48 the supernatant liquid is centrifuged at a temperature of 37 degrees Celsius until a pellet is formed. The use of this temperature facilitates separation of the microtubules from other proteins and cellular debris present in the mixture. The pellet is then discarded, and the resulting supernatant is then layered on top of a solution of buffer A containing 6 molar glycerol in step 50. Then, in step 52, this layered assembly is centrifuged again at 37 degrees Celsius for 90 minutes.

In step 54, the supernatant liquid is discarded, thereby producing a pellet that is comprised of m icrotubules. The pellet obtained in step 54 is then to isolate and purify tubulin isotypes from the microtubules.

Referring again to FIG. 2, and in step 56 thereof, various alpha- and beta-tubulin monomers are isolated from the microtubules. This isolation may be effected by the means described elsewhere in this specification, preferably by the use of immunoaffinity chromatography. This isolation technique is well known to those skilled in the art. Reference may be had, e.g., to U.S. Pat. Nos. 4,568,488 (reverse immunoaffinity chromatography purification method), 5,316,923 (immunoaffinity chromatography using specific monoclonal antibody), 5,328,604 (lignocellulosic and cellulosic beads for use in immunoaffinity chromatography of high molecular weight proteins), 5,362,857 (purification of plasminogen activator inhibitor 2 by immunoaffinity chromatography), 5,614,500 (compositions prepared by immunoaffinity chromatography), and the like. The entire disclosure of each of these United States patents is hereby incorporated by reference into this specification.

Referring again to FIG. 2, and in step 58 thereof, the tubulin isotypes that have been isolated in step 56 are thereafter purified. This purification step may be effected by conventional means, such as the immunoffinity chromatography described hereinabove and to the separation processes also described elsewhere in this specification. Thus, by way of illustration and not limitation, one may resuspend the microtubule-containing pellet in buffer A, measure the protein concentration therein, dilute the protein concentration with such buffer A, apply the diluted material to a chromatography column made of phosphocellulose that is equilibrated in buffer A, elute the chromatography column with such buffer, and recover pure tubulin from the eluent.

Referring again to FIG. 2, and in step 60—thereof, one may subject the purified tubulin(s) to a tubulin binding assay to determine how specifically such purified tubulin(s) binds to candidate drugs. Such binding assays are well known to those skilled in the art. Reference may be had, e.g., to U.S. Pat. Nos. 4,160,645 (catalyst mediated binding assay), 4,233,401 (antienzyme homogeneous competitive binding assay), 4,575,484 (binding assay for the detection of mycobacteria), 4,588,697 (fluorescent protein binding assay), 4,640,898 (homogeneous fluroescence ligand binding assay), 4,650,751 (protected binding assay avoiding non-specific protein interference), 4,839,276 (interference-resistant liposome specific binding assay), 5,413,939 (solid phase binding assay system), 5,612,455 (nucleic factors and binding assay), 5,652,111 (binding assay utilizing a nucleic acid encoding ampamin binding protein), 5,691,147 (CDK4 binding assay), 6,489,124 (human NR2A binding assay), and the like. The entire disclosure of each of these United States patent is hereby incorporated by reference into this specification.

Referring again to FIG. 2, and in step 62 thereof, the binding affinity index for a particular tubulin is then determined. Such binding affinity index may be determined, e.g., the fluorescence binding assay described in the next section of this specification. In one embodiment, the candidate drug will bind to a a beta-tubulin isotype selected from the group consisting of the class II isotype, the class III isotype, and the class V isotype at least 1.1 times as great as it binds to any other beta-tubulin isotype; it is thus said to have a binding affinity index of at least 1.1.

Fluorescence Binding Assay

The composition of this invention preferably is comprised of at least one anti-mitotic drug that preferentially binds to a beta-tubulin isotype selected from the group consisting of the class II isotype, the class m isotype, and the class V isotype.

One may may obtain tubulin biological organisms and test the degree to which candidate drugs bind to the various tubulin isotypes by means well known to those skilled in the art. Reference may be had, e.g., to U.S. Pat. Nos. 5,661,032 (Ta1 alpha-tubulin promoter and expression vectors), 5,886,025 (anti-mitotic agents which inhibit tubulin polymerization), 6,000,772 (tubulin promoter regulates gene expression in neurons), 6,162,930 (anti-mitotic agents which inhibit tubulin polymerization), 6,258,841 (tubulin binding compounds [COBRA]), 6,306,615 (detection method for monitoring beta-tubulin isotype specific modification), 6,329,420 (tubulin binding compounds [COBRA]), 6,346,389 (method for selectively modulating the interactions between surviving and tubulin), 6,350,777 (description anti-mitotic agents which inhibit tubulin polymerization), 6,433,187 (certain polycyclic compounds useful as tubulin-binding agents), 6,586,188 (method for identification of compounds that bind to beta-tubulin and stimulate insulin secretion), 6,593,374 (tubulin binding ligands and corresponding prodrug constructs), 6,676,944 (vaccine containing a perioxiredoxin and/or a beta-tubulin), 6,694,436 (B-homoestra-1,3,5(10)-trienes as modulators of tubulin polymerization), 6,750,330 (lypholized tubulins), and the like. The entire disclosure of each of these United States patents is hereby incorporated by reference into this specification.

In one preferred embodiment, the activity of certain candidate drugs is evaluated in the “fluorescence binding assay” disclosed in an article by Israr A. Khan et al., “Differential Interactio of Tubulin Isotypes with the Antimitotic Compound IKP-104,: Biochemistry 2000, 39, 9001-9009. At page 9002 of this article, certain “experimental procedures” are described.

The “experimental procedures” of the Khan et al. article relate to an assay for assessing the binding if the anti-tumour drug IKP-104 to tubulin. However, these “experimental procedures” can readily be adapted to assess the binding of other anti-tumour, anti-tubulin drugs.

As is disclosed at page 9002 of the Khan et al. reference, “Materials. GTP was purchased from Sigma Chemicals (St. Louis, Mo.). IKP-104 was synthesized at the K-I Research Institute (Shizuoka, Japan). The compound was dissolved in dimethyl sulfoxide immediately before use because repeated freezing and thawing led to a dramatic loss in its ability to bind tubulin and inhibit microtubule assembly.”

“Purification of Tubulin Isotypes. “Microtubules were prepared from bovine cerebra by the method of Fellous et al. (25); tubulin was purified therefrom by phosphocellulose chromatography.” The cited Fellous et al. reference was a 1977 publication in Eur. J. Biochem. 78, 167-174. Phosphocellulose chromatography is a well known technique; reference may be had, e.g., to U.S. Pat. Nos. 5,580,898 (method of stabilizing microtubules), 6,177,472 (regulation of alzheimer's disease proteins), 6,358,957 (phenylastin and the phenylastin analogs, a new class of anti-tumour compounds), 6,423,735 (compounds and methods for use thereof in the treatment of cancer), 6,423,736 (compounds and methods for use thereof in the treatment of cancer); 6,458,847 (method for screeining for drugs useful in inhibition of polymerization of αβ and tau peptides), 6,462,062 (compounds and methods for use thereof in the treatment of cancer); 6,482,043 (compounds and methods for use thereof in the treatment of cancer); 6,608,096 (compounds and methods for use thereof in the treatment of cancer); 6,660,767 (coumarin compounds as microtubule agents and therapeutic uses thereof), 6,710,065; 6,713,480 (phenylastin and the phenylastin analogs, a new class of anti-tumour compounds), 6,720,349 (compounds and methods for use thereof in the treatment of cancer); and the like. The entire disclosure of each of these United States patents is hereby incorporated by reference into this specification.

“Microtubule-associated proteins were prepared from the microtubules and fractionated to purify tau by gel filtration as previously described (25). The isotypically purified αβ_{II}, αβ_{III}, and αβ_{IV} dimers were prepared by immunoaffinity chromatography as described previously (26). All isotyptically purified tubulins were stored at −80° C. until they were ready for use.” The reference “25” was a 1977 Fellous et al. publication in Eur. J. Biochem. 78, 167-174. The reference “26” was an article by I. A. Khan et al. published in 1996 in Biochemistry 35, 3704-3711. The immunoaffinity chromatography technique is described, e.g., in U.S. Pat. Nos. 4,568,488 (reverse immunoaffinity chromatography); 5,316,932; 5,328,603; 5,362,857; 5,614,500; and the like. The entire disclosure of each of these United States patents is hereby incorporated by reference into this specification.

“The relative amounts of alphafbeta III in each tubulin sample were measured by subjecting the tubulin to SDS-PAGE on 5.5% gels (27).” The reference “27” was a 1970 article by U. K. Laemmli in Nature 227, 680-685. The SDS-PAGE technique is referred to, e.g., in U.S. Pat. Nos. 5,679,530 and 6,441,053, the disclosure of each of which is hereby incorporated by reference into this specification.

“Tubulin samples were reduced and carboxymethylated prior to SDS-PAGE (28). Under these conditions, the beta III isotype has an electrophoretic mobility distinctly different from those of the beta II and beta IV isotypes, which comigrate (29, 30). The immunoblotting of gels was carried out as previously described (26).” The reference “26” was an article by I. A. Khan et al. published in 1996 in Biochemistry 35, 3704-3711. The reference “29” was a 1982 article by R. F. Luduena et al. published in Biochemistry 21, 4787-4794. The reference “30” was a 1990 article by Banerjee et al. pbulisehd in J. Biol. Chem. 193, 265-275.

“Tubulin Polymerization.” The tubulin was thawed on ice-water and spun at 18000 g for 6 min at 4° C. to remove any insoluble tubulin aggregates from the sample. Tubulin present in the supernatant was quantitated by the method of Lowry et al. (31) and mixed with IKP-104 and tau in MES buffer [0.1 M MES, 1 mM GTP, 0.5 mM MgCl₂, 1 mM EDTA, 1 mM EGTA, and 1 mM β-mercaptoethanol (pH 6.4)] at 4° C./Unless otherwise mentioned, the final concentrations of tubulin and tau were 1.5 and 0.15 mg/mL, respectively. The temperature of the samples was raised from 4 to 37° C., and tubulin polymerization was followed by either sedimentation or turbidemetry as described previously (26). All the absorbance measurements were taken using a Beckman DU7400 spectrophotometer equipped with a Peliter temperature controller.” The reference “31” was a 1951 article by O. H. Lowry et al., J. Biol. Chem. 193, 265-275. The phenomenon of tubulin polymerization, and means for effecting it or inhibiting it, are known to those skilled in the art; reference may be had, e.g., to U.S. Pat. Nos. 5,886,025 (anti-mitotic agents which inhibit tubulin polymerization), 6,162,930 (anti-mitotic agents which inhibit tubulin polymerization), 6,350,777 (description anti-mitotic agents which inhibit tubulin polymerization), and 6,964,436 (B-homoestra-1,3,5(10)-trienes as modulators of tubulin polymerization). The entire disclosure of each of these United States patents is hereby incorporated by reference into this specification.

The tubulin samples were then subjected to fluorescence analysis to determine the extent to which candidate drugs interacted with the tubulin. As is disclosed in U.S. Pat. No. 6,660,767 (the entire disclosure of which is hereby incorporated by reference in to this specification), “The binding of dicoumarol was determined by taking advantage of the fluorescence properties of tubulin. Tubulin is a tryptophan containing protein. When excited, tubulin displays a typical tryptophan emission spectrum. An excitation wavelength was selected to specifically excite the tubulin tryptopanyl residues. Relative fluorescence intensities were measured and buffer blanks were subtracted from all measurements. By incubating tubulin with different concentrations of dicoumarol, whether there is concentration dependence between the binding of dicoumarol and the quenching of tubulin fluorescence was determined. See Panda et al. (1992) Eur. J. Biochem. 204:783-787; Panda et al. (1997) PNAS USA 94:10560-10564; and Panda et al (1997) J. Biol. Chem. 272:7681-7687, which are herein incorporated by reference.” Reference may also be had, e.g., to U.S. Pat. Nos. 5,851,789 and 6,472,541, the entire disclosure of which is hereby incorporated by reference into this specification.

“Electron Microscopy. The mixtures of tubulin and tau were incubated for at least 30 min. at 37° C. in the absence or presence of IKP-104 in MES buffer. The concentrations of tubulin and tau were 1.5 and 0.15 mg/mL, respectively. Aliquots (50 μL) were withdrawn and treated with 1% glutaraldehyde for 30 s, and then layered on 400-mesh copper grids coated with carbon over Formvar. After 1 min, the grids were washed with 4 drops of water and stained with 1% uranyl acetate for 1 min. Excess stain was removed, and, after air-drying, grids were examined in a JEOL 100 CX electron microscope with an accelerating voltage of 60 kv.(26).” The “26” reference was an article by I. A. Khan et al. published in 1996 in Biochemistry 35, 3704-3711.

“IKP-104 Binding Assay. Tubulin (2 μM) and IPP-104(0-25 μM) were mixed in 500 μL of 50 mM PIPES buffer (ph 7.0) containing 1 mM EGTA and 0.5 mM MgCl₂. The mixture was incubated for 30 min at 30° C., and the fluorescence intensities of the samples were recorded in a Hitachi F-2000 spectrrofluorometer. The excitation and emission wavelengths were 273 and 330 nm, respectively. The absorbance of control samples containing 0-25 μM IKP-104 (was also measured at 273 and 330 nm to correct the absorbed fluorescence intensities at 330 nm for the inner filter effect (32) The corrected absorbed fluorescence data were analyzed by using different models and equations as follows . . . wherein K_{d1}, K_{d2}, and K_{d3} are the apparent dissociation constants for the high-, low-, and lowest affinity sites, respectively.”

The fluorescence binding assay described in the 2000 Khan et al. paper is similar to an assay for assessing the binding of IKP-104 to tubulin reported in a 1998 paper by A. R. Chaudhuri et al. published in Biochemistry 37, 17157-17162. As was disclosed at page 9002 of the 2000 Khan et al. paper, the Chaudhuri et al. assay “. . . relies on the IKP-104-induced local conformational changes in tubulin and the increment of the fluorescence of IKP-104 in the IKP-104-tubulin complex at 451 nm μM. A nonlinear relationship exists between the increasing IKP-104 concentration and the resulting increase in IKP_{—}104 fluorescence. However, the fidelity of this relationship appears to exist within a narrow range of IKP-104 concentrations (0-10 μM) because at higher concentrations (e.g., >10 μM IKP-104), instead of increasing, the IKP-104 fluorescence starts decreasing . . . . In the method presented here, we have enhanced the reliability of the binding data by using a wider range of IKP-104 concentrations (0-25 μM) to obtain the near saturation of tubulin with the compound. Also, we have utilized the IKP-104-induced pertebrations in the intrinsic fluorescence of tubulin itself at 330 nm . . . as a probe for IKP=104 binding to the tubulin molecule.”

As is also reported in the 2000 Khan et al. article, there were observed “. . . differential affinities of tubulin isotypes for IKP-104 . . . ” (see page 9003). This observation was consistent with the “prior art” discussed on page 9001 of the 2000 Khan et al. article, wherein it was disclosed that “Both alpha and beta-subunits of tubulin differ as multiple isotypes . . . . The differences among the beta-isotypes, which are found mostly within the C-terminal region, have been highly conserved thoruhout evolution. Tubulin dimmers isotypically purified by their beta-subunits differ from each other in their assembly, dynamics, cellular distrubiton, post-translational modification, and conformation (13-20). The references “13” through “20” cited on this page 9001 were articles by M. A. Lopata et al. (J. Cell. Biol. 105, 1707-1720, 1987), by H. D. Hoyle et al. (J. Cell. Biol. 11, 1009-1026, 1990), by A. Banerjee et al. (J. Biol. Chem. 267, 5625-5630, 1992), by R. Renthal et al. (“Cell Motil. Cytoskeleton 25, 19-29, 1993), by D. Panda et al. (Proc. Natl. Acad. Sci. U.S.A. 91, 11358-11362, 1994), by J. S. Sharma et al. (J. Protein Chem. 13, 165-175, 1994), by M. C. Roach et al. (Cell Motil. Cytoskeleton 39, 273-285, 1998), and by P. M. Schwartz et al. (Biochemistry 37, 4687-4692, 1998).

As is also disclosed on page 9001 of the 2000 Khan et al. article, “Some of the most interesting differences among the isotypes involve their interactions with ligands.The alpha/beta III dimer interacts much less strongly with colchicines, vinblastine, and paclitaxel than do the alpha/beta II and alpha/beta IV dimers (21-24).” The references cited as “21-24” were articles by B. Banerjee et al. (J. Biol. Chem. 267, 13335-13339, 1992), by W. B. Derry et al. (Biochemistry 36, 3554-3562, 1997), and by I. A. Khan et al. (a 1994 publication in Mol. Biol. Cell. 5, 284a; and a 1995 publication in Mol. Biol. Cell 6, 30a).

In one preferred embodiment of the instant invention, an alpha/beta dimer selected from the group consisting of alpha/beta II dimer, alpha/beta III, and alpha/beta V dimer is tested with candidate anti-tubulin agents at the “LC₅₀” concentrations. As is known to those skilled in the art, the “LC₅₀” is the concentration of the toxic compound that is lethal to 50% of the organism to be tested under the test conditions in a specifed time. It is often also referred to as the “lethal concentration” or the “median lethal dose.” Reference may be had, e.g., to U.S. Pat. Nos. 5,470,8222 (low-toxicity invert emulsion fluids), 5,549,840, 5,593,958, 5,599,785, 5,731,281, 5,827,679, and the like. The entire disclosure of each of these United States patents is hereby incorporated by reference into this specification.

In this embodiment, the apparent dissociation constant for the high affinity site of the tubulin isotype in question is determined. Without wishing to be bound to any particular theory, applicant believes that the interaction of a drug with the its receptors is considered to follow what is termed “mass action kinetics”—the interaction follows the Law of Mass Action, which requires the rates of chemical processes to relate systematically to the concentrations of the interacting compounds. Thus, if A+B interact to form AB, then the rate of the forward (f) reaction is dependant on a rate constant, kf multiplied by the product of the concentrations A and B (Rate of forward reaction=kf [A] [B]). By the same standard, the reverse (r) reaction is dependant on a rate constant, kr multiplied by the concentration of AB (Rate of reverse action=kr [AB]). At at equilibrium the rates of the forward and reverse reactions are equal.

The ratio kr divided by kf, is the equilibrium, or dissociation constant, commonly given the symbol Kd.

In the interaction of drugs with the protein tubulin, it is known that there are three binding sites, designated high affinity binding sites, low affinity binding sites, and very low affinity binding sites. The dissociation constants for interaction of drugs with these binding sites are given the symbols K_{d1}, K_{d2}, and K_{d3} respectively. As is described in, e.g., the 2000 Khan et al. article, the rates of reaction, k and K values are studied and measured using fluorescence spectroscopy with excitation and emission wavelengths of 273 and 330 nm respectively, and mathematical analysis of the data generated.

Any particular drug will have a characteristic set of values for these three dissociation constants when interacting with a particular tubulin isotype. Thus a unique set of dissociation constant ratios can be sought in the search for optimized interaction with, e.g., certain tubulin isotypes.

Referring again to FIG. 2, and to step 62 thereof, for any particular isotypically purified dimer selected from the group consisting of the alpha/beta IIdimer, the alpha/beta III dimer, and the and alpha/beta V dimer, the apparent dissociation constant ratio for the dimer is at least 1.5 and, more preferably, at least 2.0. The apparent dissocation constant ratio is the ratio of the apparent dissociation constant (Kd1) for the dimer selected from the group consisting of the alpha/beta II dimer, the alpha/beta II dimer, and the and alpha/beta V dimer,divided by the highest apparent dissociaton constant (Kd1) for a dimer selected from the group consisting of the alpha/beta I dimer, the alpha/beta IV dimer, and the alpha/beta VI dimer.

Referring to page 9003 of the 2000 Khan article, it should be noted that, with regard to the alpha/beta III dimer, IKP-104 achieved an opposite result. As is disclosed on such page 9003, “The apparent dissociation constant for the high-affinity site (K_{d1}) on alpha/beta IV was greater than those of unfractionated tubulin, alpha/beta II, or alpha/beta III Both alpha/beta II and alpha/beta IIIhad K_{d1} values that were lower than that of unfractionated tubulin.”

In one embodiment, the K_{d1} value for a dimer selected from the group consisting of the αβ_{II} dimer, the αβ_{III} dimer, and the and αβ_{V} dimer is at least 1.1 times as great as the K_{d1} value for unfractionated tubulin; in one embodiment, it is at least about 2.0 times as great as the K_{d1} value for unfractionated tubulin.

Referring again to FIG. 2, and in step 64 thereof, the therapeutic indices of those candidate compositions that pass the binding affinity tests are determined. One may determine the therapeutic index by means well known to those skilled in the art. Reference may be had, e.g., to U.S. Pat. Nos. 5,830,452 (method for enhancing the anti-tumor therapeutic index of interleukin-2), 6,222,093 (methods for determining therapeutic index from gene expression profiles), and the like. The entire disclosure of each of these United States patents is hereby incorporated by reference into this specification.

As is known to those skilled in the art, the therapeutic index (TI) assesses the margin of safety associated with the use of a drug. It can be measured in a variety of ways, in vitro and in vivo. For example, it can be expressed as: TI=IC50 in vitro killing non-cancer cells/IC50 in vitro killing cancer cells; and such index is preferably at least about 1.1 and more preferably at least about 2. In one embodiment, such index is at least about 5.0. In yet another embodiment, the therapeutic index is at least about 10.

As is known to those skilled in the art, the IC50 the concentration that inhibits growth of, or kills, 50% of the cells in a particular population in defined conditions (such as particular incubation medium, pH, temperature etc.). Reference may be had, e.g., to U.S. Pat. Nos. 5,466,620 (immunoassays for insulin sensitive enhancers), 6,025,330 (inhibitors of fibrin cross-linking and/or transglutaminases), 6,232,089 (CD23 processing enzyme preparation), 6,346,408 (method of allophycocyanin inhibition of enterovirus), 6,451,807 (methods of treating sexual dysfunction using a PDE5 inhibitor), 6,576,219 (method for enhancing outflow of aqueous humor in treatment of glaucoma), 6,620,818 (method for reducing the severity of side effects of chemotherapy), 6,630,492 (lymphocyte function antigen-1 antagonists), 6,635,434 (immunoassay for pesticides and their degradation products), and the like. The entire disclosure of each of these United States patents is hereby incorporated by reference into this specification.

The IC50 derives from the sigmoid (S-shaped) graph of response (expressed as percent of cells inhibited) vs. log. concentration. The IC50 is the antilog. of the logarithm of the concentration of drug associated with a 50% response.

The therapeutic index can also be expressed as the ratio of any convenient measure of toxicity (or side effect) of a drug to any convenient measure of the desired effect of that drug in a laboratory animal such as the mouse. For example, the drug might have antimitotic activity at x mg/kg, but cause respiratory paralysis at y mg/kg. In this case: TI=y/x.

Similarly, the therapeutic index can be measured in humans. For example, the drug of the previous paragraph might cause a skin rash in humans at, say, 2y mg/kg, but need only 0.5x mg/kg to kill tumors, and not cause respiratory paralysis in humans. In this case: TI=2y/0.5x=4y/x. Thus the TI will be a different number depending on the circumstances of its measurement, in vitro, in animals or in humans. Generally speaking, a drug will need at least a ten-fold margin of safety (TI) in humans in vivo, and while other measurements of TI may be important in the process of drug discovery, only the human, in vivo, in patients, TI is of significance in therapeutics. Our goal is an infinitely high TI, and this may be attainable with highly selective tubulin inhibition.

As is obvious from the description provided hereinabove, one may evaluate candidate drugs to determine their suppressivity with various tubulin dimers comprised of an alpha isotype and a beta isotype. It is preferred that the alpha isotypes be selected from the group consisting of human alpha 1, human alpha 2, human alpha 3, human alpha 4, human alpha 6, human alpha 8, and the human equivalent of mouse alpha TT1. It is preferred that the beta isotype be selected from the group consisting of human class IA, human class 1b, human class 2, human class 3, human class 4a, human class 4b, human class 5, human class 6, and human class 7.

Purification of alpha/beta tubulin dimers has been discussed elsewhere in this specification. In one embodiment, isotypically purified αβ_{II}, αβ_{III}, αβ_{IV}, and αβ_{V} dimers are preferably prepared by immunoaffinity chromatography in substantial accordance with the method described in a 1996 article by I. A. Khan et al. (Biochemistry 35, 3704-3711). Immunoaffinity chromatography is a well known process and is described, e.g., in U.S. Pat. Nos. 4,568,488 (reverse immunoaffinity chromatography purification method), 5,316,932 (homogeneous denatured human 06-guanine alkyl transferase prepared by immunoaffinity chromoatography using monoclonal antibody specific for enzyme), 5,328,603 (lignocellulosic and cellulosic beads for use in affinity and immunoffinity chromatography of high molecular weight proteins), 5,362,857 (purification of plasminogen activator inhibitor 2 by immunoaffinity chromatography), 5,614,500 (compositons containing highly purified factor IX proteins purified by immunaffinity chromatography), and the like. The entire disclosure of each of these United States patents is hereby incorporated by reference into this specification.

The differences among the tubulin isotypes are functionally significant, and thus the purified isotypes behave differently from each other in vitro. As has been discussed elsewhere in this specification, monoclonal antibodies have been constructed specific for the mammalian betaI, betaII, betaIII and betaIV isotypes, and they have been used to purify the alpha/betaII, alpha/betaIII and alpha/betaIV dimers from bovine brain by immunoaffinity chromatography. A large number of parameters have been assayed in vitro. The dimers differ from each other in virtually every parameter that has been assayed.

Assembly into microtubules is an obvious first parameter that has been examined. In the presence of either tau or MAP2, alpha/betaII and alpha/betaIII assemble more rapidly and to a greater extent than does alpha/betaIV See, e.g., the article by A. Banerjee et al., “Preparation of a monoclonal antibody specific for the class IV isotype of beta-tubulin. Purification and assembly of alpha/beta II, alpha/beta III, and alpha/beta IV tubulin dimers from bovine brain,”. J. Biol. Chem. 267, 5625-5630.

In the absence of MAPs, but in the presence of 4 M glycerol, alpha/betaII and alpha/betaIV assemble rapidly with no lag time, while alpha/betaIII assembles only after a considerable lag time. See, e.g., an article by Q. Lu et al., “In vitro analysis of microtubule assembly of isotypically pure tubulin dimers. Intrinsic differences in the assembly properties of alpha/beta II, alpha/beta III, and alpha/beta IV tubulin dimers in the absence of microtubule-associated proteins, J. Biol. Chem. 269, 2041-2047, 1994.

Microtubules formed from abIII are considerably more dynamic than those formed from either alpha/beta II or alpha/beta IV (see a 1994 article by D. Panda et al. on “Microtubule dynamics in vitro are regulated by the tubulin isotype composition,”. Proc. Nat. Acad. Sci. USA 91, 1158-1362). Possibly consistent with these findings is that the intrinsic GTPase activity of tubulin is highest for alpha/beta III than for either alpha/beta II or alpha/beta IV (see a 1997 article by A. Banerjee, “Differential effects of colchicine and its B-ring modified analog MTPT on the assembly-independent GTPase activity of purified β-tubulin isoforms from bovine brain,” Biochem. Biophys. Res. Commun. 231, 698-700). However, during microtubule assembly in the absence of MAPs, alpha/betalll hydrolyzes GTP more slowly than do the other two dimers (see a 1998 article by Q. Lu et al., “Structural and functional properties of tubulin isotypes,”. Adv. Struct. Biol. 5, 203-227).

Structural differences among the isotypes are also evident. For example, the mammalian bIII isotype is phosphorylated, while the others are not. See a 1996 article by I. A. Khan et al., “Phosphorylation of beta III-tubulin,” Biochemistry 35, 3704-3711.

Using differential scanning calorimetry, Schwarz et al. (1998, “beta-Tubulin isotypes purified from bovine brain have different relative stabilities,” Biochemistry 37, 4687-4692) found that alpha/betaIII is considerably more resistant to decay than is alpha/beta II. The half-times for decay at 37° C. of colchicine-binding activity for alpha/beta II and alpha/betaIII were, respectively, 17 hours and 50 hours

The isotypes also differ in their ligand-binding properties. This has been studied in greatest detail with colchicine and its analogues. Colchicine binds to tubulin in a slow, irreversible, and temperature-dependent manner Reference may be had to a 1965 article by E. W. Taylor (“The mechanism of colchicine binding inhibition of mitosis I. Kinetics of inhibition and the binding of H³-colchicine.”. J. Cell Biol. 25, 145-160), a 1966 article by L. Wilson et al. (“The biochemical events of mitosis. I. Synthesis and properties of colchicine labeled with tritium in its acetyl moiety,”. Biochemistry 5, 2463-2468), a 1967 article by G. G. Borsiy et al. (“The mechanism of action of colchicine: Binding of colchicine-³H to cellular protein,”. J. Cell Biol. 34, 525-533), a 1968 article by R. C. Weisenberg et al. (“The colchicine-binding protein of mammalian brain and its relation to microtubules. Biochemistry 7, 4466-4479), a 1970 article by L. Wilson (“Properties of colchicine-binding protein from chick embryo brain. Interactions with Vinca alkaloids and podophyllotoxin,” Biochemistry 9, 4999-5007), a 1973 article by L. Wilson (“The mechanism of action of colchicine: colchicine-binding properties of sea urchin sperm tail outer doublet tubulin,”. J. Cell Biol. 58, 709-714), and a 1974 article by L. Wilson et al. (“Biochemical and pharmacological properties of microtubules,” Adv. Cell Mol. Biol. 3, 21-72).

As has been discussed elsewhere in this specification in greater detail, the binding of drug to tubulin results in a promotion of drug fluorescence that has been used to characterize this interaction. Reference may be had, e.g., to articles by B. Bhattacharyya et al. (“Promotion of fluorescence upon binding of colchicine to tubulin,” Proc. Nat. Acad. Sci. USA 71, 2627-2631, 1974), by T. Arai et al. (“Fluorometric assay of tubulin-colchicine complex,” Anal. Biochem. 69, 443-448, 1975)

The binding of colchicines to tubulin is a two-step process in which initial complex formation is followed by a slow conformational change resulting in the formation of a stable complex (see articles by D. Garland [“Kinetics and mechanism of colchicine binding to tubulin: evidence for ligand-induced conformational change,”. Biochemistry 17, 4266-4272, 1978] and A. Lambeir et al. [“A fluorescence stopped flow study of colchicine binding to tubulin,” J. Biol. Chem. 256, 3279-3282, 1981]). When the association kinetics are studied under pseudo-first-order conditions, the kinetics exhibit a biphasic pattern. Biphasic kinetics are also observed for the faster-binding analogs of colchicine such as desacetamidocolchicine (DAAC) and the bicyclic analogue 5-(2′,3′,4′-trimethoxyphenyl)-1-methoxytropone (MTPT), which binds to tubulin almost instantaneously.

The origin of the biphasic kinetics in the colchicine-tubulin interaction was not clear until it was demonstrated that immunoaffinity depletion of the tubulin dimers to remove the alpha/betaIII dimer eliminated the slow phase, resulting in monophasic kinetics. Furthermore, addition of alpha/betaIII to the alpha/betaIII-depleted tubulin restored the biphasic kinetics. Subsequent kinetic studies with the isotypically pure tubulin dimers demonstrated that the isotypes differ significantly in their on-rate constants for binding colchicine. Scatchard analysis revealed that the isotypes also differ in their affinity constants for colchicine and its B-ring analogues. Reference may be had to 1994 and 1997 articles by Banerjee et al. on “Interaction of desacetamidocolchicine, a fast-binding analogue of colchicine with isotypically pure tubulin dimers αβ_{II}, αβ_{III}, and αβ_{IV}. J.,” Biol. Chem. 269, 10324-10329, and on “Interaction of a bicyclic analogue of colchicine with purified β-tubulin isoforms from bovine brain,” Eur. J. Biochem. 246, 420-424.

Analysis of the binding kinetics of colchicine and its analogues indicated that, not only does alpha/betaIII have the lowest affinity for colchicine, but that the rate (k2) of the conformational change in tubulin that is part of the drug binding reaction is slowest for alpha/betaIII (see the aforementioned 1994 and 1997 Banerjee et al. articles). The slow rate of this conformational change may reflect the greater rigidity of alpha/betaIII. If this is the case, then this may explain its lessened ability to interact with nocodazole and taxol, although the binding kinetics of these drugs with tubulin isotypes have not been studied in any detail.”

The interaction of Vinca alkaloids with purified tubulin isotypes is more complicated. One study compared the effects of vinblastine on abII, abIII, and abIV and measured vinblastine's ability to inhibit microtubule assembly and induce spiral aggregate formation (see a 2003 article by I. A. Khan et al. on “Different effects of vinblastine on the polymerization of isotypically purified tubulins from bovine brain,” Invest. New Drugs 21, 3-13). The results were clear: microtubule assembly of abIII was least sensitive to inhibition by vinblastine. Similarly, abIII was the least susceptible to vinblastine-induced aggregation. Interestingly, although vinblastine induced abIV to form spiral aggregates, abIII generally formed amorphous aggregates instead.

A startling difference in vinblastine-induced aggregation was seen when vinblastine (20 mM) was added to preparations of erythrocyte tubulin and brain tubulin from chickens (see an article by R. F. Ludueña et al., “Different activities of brain and erythrocyte tubulins toward a sulfhydryl group-directed reagent that inhibits microtubule assembly,”. J. Biol. Chem. 260, 1257-1264, 1985). The former consists largely of abVI, while the latter is likely to be a mixture of abI, abII, abIII and abIV (95). About 42% of the brain tubulin aggregated into spirals while 74% of the erythrocyte tubulin formed spirals. Aggregation of the latter was so dramatic that the resulting flocculent precipitate was readily visible to the naked eye. Clearly, abVI has a unique ability to interact with vinblastine. Conceivably, the ability of bVI to form microtubules in which the protofilaments bend so as to form a circular microtubule may translate into a greater ability for the protofilaments to bend to form the vinblastine-induced spiral.

The number of proteins or other factors known to interact with tubulin is rising very quickly. To name but a few, in addition to the well-known MAPs, we have various chaperones (see articles by A. Guasch et al. [“Three-dimensional structure of human tubulin chaperone cofactor,” A. J. Mol. Biol. 318, 1139-1149, 2002]. by Y. Saito et al. [“Identification of α-tubulin as an hsp105α-binding protein by the yeast two-hybrid system,” Exp. Cell Res. 286, 23-240, 2003]), the collapsin-response mediator protein 2 (see an article by Y. Fukata et al., “CRMP-2 binds to tubulin heterodimers to promote microtubule assembly,”. Nature Cell Biol. 4, 583-591, 2002), stable-tubulin-only polypeptide/(see an article by C. Bonnet et al., “Interaction of STOP with neuronal tubulin is independent of polyglutamylation,” Biochem. Biophys. Res. Commun. 297, 787-793, 2002), the importin/Ran-GTP system (see an article by Ems-McClung et al., “Importin α/β and Ran-GTP regulate XCTK2 microtubule binding through a bipartite nuclear localization signal,” Mol. Biol. Cell 15, 46-57, 2004), XMAP215 (see an article by K. Kinoshita et al., “XMAP215: a key component of the dynamic microtubule cytoskeleton,”. Trends Cell Biol. 12, 267-273, 2002), Fhit (See A. R. Chaudhuri et al., “The tumor suppressor protein Fhit. A novel interaction with tubulin,” J. Biol. Chem. 274, 24738-24382, 1999), katanin (See C. Lu et al., “The Caenorhabditis elegans microtubule-severing complex MEI-1/MEI-2 katanin interacts differently with two superficially redundant β-tubulin isotypes,” Mol. Biol. Cell 15, 142-150, 2004), aurora kinase (See M. Murata-Hori et al., “Probing the dynamics and functions of aurora B kinase in living cells during mitosis and cytokinesis,”. Mol. Biol. Cell 13, 1099-1108, 2002), stathmin (See P. Curmi et al., P., Andersen, S. S. L., Lachkar, S., Gavet, O., Karsenti, E., Knossow, M., et al., “The stathmin/tubulin interaction in vitro,” J. Biol. Chem. 272, 25029-25036, 1997), clathrin-coated vesicles (see J. Z. Rappoport et al., “Movement of plasma-membrane-associated clathrin spots along the ricrotubule cytoskeleton,”. Traffic 4, 460-467, 2003), aggregosomes (see R. Garcia-Mata et al., “Hassles with taking out the garbage: aggravating aggresomes,” Traffic 3, 388-396, 2002), and the proteins of the axoneme, centrosome, and basal body (see S. Geimer et al., “The ultrastructure of the Chlamydomonas reinhardtii basal apparatus: identification of an early marker of radial asymmetry inherent in the basal body,”. J. Cell Sci. 117, 2663-2674, 2004; also see K. Matsuura et al., “Bld10p, a novel protein essential for basal body assembly in Chlamydomonas: localization to the cartwheel, the first ninefold symmetrical structure appearing during assembly,”. J. Cell Biol. 165, 663-671, 2004)

In addition to mitosis and the other classical microtubule functions, microtubules are thought to be involved in processes such as determination of neuronal polarity and intra-manchette transport. Reference may be had, e.g., to articles by P. W. Baas (“Neuronal polarity: microtubules strike back,”. Nature Cell Biol. 4, E194-E195, 2002), and by A. L. Kierszenbaum et al., A. L., “Intramanchette transport (IMT): managing the making of the spermatid head, centrosome, and tail,”. Mol. Reprod. Devel. 63, 1-4, 2002).

In one preferred embodiment, the compositon of this invention contains at least a first anti-mitotic agent that, with regard to one (a first) of the dimers selected from the group consisting the alphalbeta II dimer, the alphalbeta III dimer, and the and alpha/beta V dimer, has K_{d1} value that is at least 1.1 times as great (and preferably at least 1.5 times as great) as (a) the K_{d1} value for unfractionated tubulin, and (b) the highest K_{d1} value for a dimer selected from the group consisting the alpha/beta I dimer, the alphalbeta IV dimer, and the and alpha/beta VI dimer. As will be apparent, such first antimitotic agent will preferentially bind to and inactivate a dimer selected from the group consisting the alpha/beta II dimer, the alpha/beta III dimer, and the and alpha/beta V dimer.

In another embodiment of the invention, the composition of this invention also preferably contains a second anti-mitotic agent that with regard to a separate (a second) dimer selected from the group consisting of the alpha/beta II dimer, the alpha/beta III dimer, and the αβ_{V} dimer has K_{d1} value that is at least 1.1 times as great (and preferably at least 1.5 times as great) as (a) the K_{d1} value for unfractionated tubulin, and (b) the highest K_{d1} value for a dimer selected from the group consisting the alpha/beta I dimer, the alpha/beta IV dimer, and the and alpha/beta IV dimer. As will be apparent, such first antimitotic agent will preferentially bind to and inactivate such second dimer selected from the group consisting the alpha/beta II dimer, the alpha/beta III dimer, and the and alpha/beta V dimer.

Synergistically Effective Combinations of the Two Antimitotic Agents

In one preferred embodiment, the first antimitotic agent, and the second antimitotic agent, are preferably present in synergistically effective amounts, i.e., in amounts such that the therapeutic index of the two-agent composition at a given degree of efficacy is preferably lower than the therapeutic index of either drug when used by itself. For a discussion of such “synergistically effective” effects, reference may be had, e.g., to U.S. Pat. Nos. 4,863,727 (combination therapy using interleukin-2 and tumor necrosis factor); 4,628,063; 4,730,007; 4,791,101 (synergistic mixtures of interferons and tumor necrosis factor); 5,073,492 (synergistic combination for endothelial cell growth); 5,098,702 (combination therapy using interleukin-2 and tumor necrosis factor), 5,204,329; 5,242,909; 5,425,940; 5,514,664; 5,654,328 (method for reducing tumor development with a combination of platinum and tellurium compounds); 5,677,331; 5,679,978 (methods of inhibiting undesirable cell growth using a combination of a cylocreatine compound and a hyperplastic inhibitory agent); 5,698,214; 5,728,687; 5,981,536; 6,063,780; 6,083,919; 6,121,263; 6,147,060 (treatment of carcinomas uwing squalamine in combination with other anti-cancer agents); 6,267,949; 6,277,835; 6,319,923; 6,403,562; 6,514,949; 6,514,960; 6,518,278; 6,528,515 (combination therapy to treat hepatitis B virus); 6.596,712 (treatment of carcinomas using squalamine in combination with other anti-cancer agents); 6,602,870; 6,617,333 (antineoplastic combinations); 6,627,649; 6,689,762; 6,693,125 (combinations of drugs for treatment of neoplastic disorders); 6,703,380; 6,762,174 (low molecular weight compounds administered together with anti-cancer agents to prevent or treat cancer); 6,747,036; and the like. The entire disclosure of each of these United States patents is hereby incorporated by reference into this specification.

Referring again to FIG. 2, and in step 66 thereof, the clonogenic survival of those compositons that have passed the therapeutic index test and the tubulin binding specificity test are determined, preferably by conventional means.

The composition of this invention preferably has clonogenic survival of less than about 0.1 percent. As is known to those skilled in the art, clonogenic survival is the ability of a single cell to give rise to a colony of cells on a Petri plate. Reference may be had to a text by Edward L. Alpen, “Radiation Biophysics” (Academic Press, 2d edition, 1998), pages 132 et seq. Reference may also be had to, e.g., U.S. Pat. No. 5,965,126 (see claim 9), the entire disclosure of which is hereby incorporated by reference into this specification.

The term clonogenic survival does not describe the continued existence of a single cell, but rather describes the ability of a cell to reproduce. Thus, and referring to pages 132-133 of the Alpen text, “This end point is sometimes referred to as reproductive death, as distinguished from true survival, which is the continued functional and metabolic existence of one cell.”

In one preferred embodiment, the composition of this invention is comprised of at least two distinct anti-mitotic agents, each of which has an anti-mitotic index of at least about 10 percent and, more preferably, at least about 20 percent. In one aspect of this embodiment, the mitotic index factor is at least about 30 percent. In another embodiment, the mitotic index factor is at least about 50 percent.

In another embodiment of the invention, the composition of this invention has a mitotic index factor of less than about 5 percent.

As is known to those skilled in the art, the mitotic index is a measure of the extent of mitosis. Reference may be had, e.g., to U.S. Pat. Nos. 5,262,409 (binary tumor therapy), 5,443,962 (methods of indentifying inhibitors of cdc25 phosphatase), 5,744,300 (methods and reagents for the indentificatioin and regulation of senescence-related genes), 6,613,318, 6,251,585 (assay and reagents for indentifying anti-proliferative agents), 6,252,058 (sequences for targeting metastatic cells), 6,387,642 (method for indentifying a reagent that modulates Myt1 activity), 6,413,735 (method of screening for a modulator of angiogenesis), 6,531,479 (anti-cancer compounds), 6,599,694 (method of characterizing potential therapeutics by determining cell-cell interactions), 6,620,403 (in vivo chemosensitivity screen for human tumors), 6,699,854 (anti-cancer compounds), 6,743,576 (database system for predictive cellular bioinformatics), and the like. The entire disclosure of each of these United States patents is hereby incorporated by reference into this specification.

Reference may also be had, e.g., to U.S. Pat. No. 5,262,409, which discloses that: “Determination of mitotic index: For testing mitotic blockage with nocodazole and taxol, cells were grown a minimum of 16 hours on polylysinecoated glass coverslips before drug treatment. Cells were fixed at intervals, stained with antibodies to detect lamin B, and counterstained with propidium iodide to assay chromosome condensation. To test cell cycle blocks in interphase, cells were synchronized in mitosis by addition of nocodazole (Sigma Chemical Co.) to a final concentration of 0.05 μg/ml from a 1 mg/ml stock in dimethylsulfoxide. After 12 hours arrest, the mitotic subpopulation was isolated by shakeoff from the culture plate. After applying cell cycle blocking drugs and/or 2-AP, cells were fixed at intervals, prepared for indirect immunofluorescence with anti-tubulin antibodies, and counterstained with propidium iodide. All data timepoints represent averages of three counts of greater than 150 cells each. Standard deviation was never more than 1.5% on the ordinate scale.”

Reference may be had, e.g., to U.S. Pat. No. 6,413,735 which discloses that: “The mitotic index is determined according to procedures standard in the art. Keram et al., Cancer Genet. Cytogenet. 55:235 (1991). Harvested cells are fixed in methanol:acetic acid (3:1, v:v), counted, and resuspended at 106 cells/ml in fixative. Ten microliters of this suspension is placed on a slide, dried, and treated with Giemsa stain. The cells in metaphase are counted under a light microscope, and the mitotic index is calculated by dividing the number of metaphase cells by the total number of cells on the slide. Statistical analysis of comparisons of mitotic indices is performed using the 2-sided paired t-test.”

By means of yet further illustration, one may measure the mitotic index by means of the procedures described in, e.g., articles by Keila Torres et al. (“Mechanisms of Taxol-Induced Cell Death are Concentration Dependent,” Cancer Research 58, 3620-3626, Aug. 15, 1998), and Jie-Gung Chen et al. (“Differential Mitosis Responses to Microtubule-stabilizing and destablilizng Drugs,” Cancer Research 62, 1935-1938, Apr. 1, 2002).

The mitotic index is preferably measured by using the well-known HeLa cell lines. As is known to those skilled in the art, HeLa cells are cells that have been derived from a human carcinoma of the cervix from a patient named Henrietta Lack; the cells have been maintained in tissued culture since 1953.

Hela cells are described, e.g., in U.S. Pat. Nos. 5,811,282 (cell lines useful for detection of human immunodeficiency virus), 5,376,525 (method for the detectioin of mycoplasma), 6,143,512, 6,326,196, 6,365,394 (cell lines and constructs useful in production of E-1 deleted adenoviruses), 6,440,658 (assay method for determining effect on aenovirus infection of Hela cells), 6,461,809 (method of improving inflectivity of cells for viruses), 6,596,535, 6,605,426, 6,610,493 (screening compounds for the ability to alter the production of amyloid-beta-peptide), 6,699,851 (cytotoxic compounds and their use), and the like; the entire disclosure of each of these United States patents is hereby incorporated by reference into this specification. By way of illustration, U.S. Pat. No. 6,440,658 This patent discloses that, for the experiments described in such patent, “The HeLa cell line was obtained from the American Type Culture Collection, Manassas Va.”

In one preferred embodiment, the mitotic index of a “control cell line” (i.e., one that omits that drug to be tested) and of a cell line that includes 50 nanomoles of such drug per liter of the cell line are determined and compared. The “mitotic index factor” is equal to (Mt−Mc/Mc)×100, wherein Mc is the mitotic index of the “control cell line,” and Mt is the mitotic index of the cell line that includes the drug to be tested.

In one embodiment, preferably wherein the composition is comprised of at least two anti-mitotic agents, at least one of the anti-mitotic agents in the composition is a thioredoxin reductase inhibitor. This inhibitor is described elsewhere in this specification.

In yet another embodiment, at least one of the anti-mitotic agents, and preferably at least two of the anti-mitotic agents, is an anti-tubulin drug. These drugs and method for identifying them, are disclosed, e.g., in U.S. Pat. No. 6,500,405 for “use of certain amides as probes for detection of antitubulin activity and resistance monitoring.” As will be apparent, one may use the process of this patnt do determine the extent to which any particular drug inactivates tubulin.

In one preferred embodiment, a stable transformed cell line is obtained. Stable transformed cell lines may be commercially obtained from e.g., the ATCC of Manassas, Va. Suitable transformed cell lines include, e.g., MCF-7 (breast cancer), HeLa (ovarian cancer cell line) C6 (brain gliosarcoma cell line), PC3 (prostate cancer cell line), SKOV3 (ovarian cancer cell line), CHO (ovariancancer cell line), and the like. For purposes of discussion, the use of the MCF-7 cell line is discussed line.

Primary cell lines may be derived from biopsies of cancerous tissue from patients. In one embodiment, the biopsies are conducted of breast cancerous tissue.

In one preferred embodiment of the invention, the amino acid sequences of the purified isotypes are evaluated to determine their degree of sequence identity with each other. Those isotypes that have less than about 90 percent sequence identity with the other isotypes are excellent candidates for drugs that will specifically bind to them.

As will be apparent, by identifying those tubulin isotypes in a particle cell that have less than about 90 percent of amino acid sequence homology, one can readily identify targets for drugs will selectively attack such isotypes and no others. By the same token, one can identify tubulin dimers (each of which is comprised of a separate alpha co-monomer and a beta subunit) and, thereafter, identity that tubulin dimers which have less than about 90 percent of amino acid sequence homology with the other tubulin dimers in the cell. Put another way, one can identify dimers whose alphas have a sequence identity to other alphas, and whose betas have a sequence identity to other betas that, on average, is less than about 90%.

In one embodiment, a beta-tubulin specific drug is used that combats malaria. As is known to those skilled in the art, every eukaryotic organism has tubulin in it. These tubulin-containing organisms include several groups of disease causing organisms, including worms, fungi, and protists. Knowing the sequences of these various pathogens, one can design drugs specific for these diseases. The pathogens one could target include but are not limited to the ones named here followed by the diseases in parentheses: Echinococcus (tapework infestation), Schistosoma (schistosomiasis, bilharzia), Histoplasma (histoplasmosis), Aspergillus (aspergillosis), Candida (candidiasis), Trichoderma (trichodermiasis), Erisyphe (grass mildew), Pneumocystis (pneumocystis), Cryptococcus (cryptococcidiosis), Encephalitozoon (encephalitis), Cryptosporidium (cryptosporidiosis), Toxoplasma (toxoplasmosis), Babesia (babesiosis), Plasmodium (malaria), Leishmania (leishmaniasis, kala azar, Bagdad boil), Giardia (giardiasis), Trypanosoma (trypanosomiasis, sleeping sickness, Chagas disease), and Trichomonas (trichomoniasis).

A Blood Test to Determine the Presence of Cancer

The current “best test” for the presence of prostate cancer is a blood test called the Prostate Specific Antigen (PSA) test, which measures the level of the PSA glycoprotein in the blood of a patient. Growing evidence has shown that the test is inaccurate, resulting in a false positive test result of as much as 75% (Barry M. J. Prostate-specific antigen testing for early diagnosis of prostate cancer. N. Engl. J. Med., 344: 1373-1377, 2001.) The prostate normally becomes enlarged in men as they grow older, resulting in an increase of the level serum PSA with age. This increase can mislead to a clinician to conclude that the follow up confirmatory procedure, a surgical biopsy of the prostate be performed. As described by K. Ito et al. in Urology (Vol. 62, Issue 1, July 2003, Pages 64-69), “Natural history of PSA increase with and without prostate cancer”: “The probability of a “non-cancer-related increase,” “suspicious cancer-related PSA increase,” and “cancer-related PSA increase” was 57%, 15%, and 28%, respectively. The PSA velocity before the PSA increase was not significantly different between those with and without prostate cancer. A “non-cancer-related PSA increase” was observed in 92% of those with a PSA increase within 2 years of baseline PSA ranges of 2.0 ng/mL or less. Regardless of elapsed years until a PSA increase to greater than 4.0 ng/mL, a “suspicious cancer-related PSA increase” or “cancer-related PSA increase” was observed in almost one half of those with baseline PSA levels of 2.1 to 4.0 ng/mL.” Further, Dr. Ito states: “intensive serial observations should be recommended before undergoing biopsy for those with a PSA increase within 2 years of a baseline PSA range of 0.0 to 2.0 ng/mL. It may be difficult to distinguish between those with and without cancer using only subsequent total PSA measurements for the remaining cases, and prostate biopsy should be recommended at present.” This biopsy procedure is costly and uncomfortable for the patient and avoiding it would be a benefit to the patient and clinician. As is alluded to by Dr. Ito, the PSA test can vary significantly from patient to patient and no diagnosis can be conclusive from any single result.

As previously described in this application, the beta II isotype of tubulin has been found in the nuclei of cancerous cells and not in the nuclei of normal cells. The exception to this is that it has been found that cells of the immune systems in the proximity of these beta II containing cells have been found also to have beta II in their nuclei. The circulating cells of the immune system, however, may serve as an additional indicator for cancer diagnosis.

By isolating a single cell type, and then focusing interrogation of the cell to a single organelle (the nucleus), a specific and reliable test is produced. Exploiting the unique and cancer specific distribution of this normal and unmutated genetic material is a more accurate test than is currently available. This disclosure describes a test that can identify tubulin mRNA localized to the nuclei with a preferred embodiment in which the Polymerase Chain Reaction (PCR) or reverse-transcription PCR (rtPCR) use oligonucleotide primers specific for tubulin beta II mRNA. Addtionally, other primers that could be used in this test include, but are not limited to, those specific for sequences of beta III, beta IV, and beta V mRNA.

As described by Ito H, et al. in “Detection and quantification of circulating tumor cells in patients with esophageal cancer by real-time polymerase chain reaction” (J Exp Clin Cancer Res. 2004 September; 23(3):455-64), cells of cancer tissues often enter the blood stream in a process of epidermal sluffing, a process in which the cells of the cancer non-specifically detach from the tumor mass and circulating in the blood stream. In addition, the invasive process “metastasis”, in which cells from a main tumor mass break off and travel in the body, allows cells from the tissue tumor to enter the bloodstream. These metastatic cells can take up residence in parts of the body distant from its point of origin and grow into a separate tumor mass. Both of these types of cells can be isolated and detected as described by the process described.

As described previously in this specification, Ranganathan et al. (“Immunohistochemical analysis of beta-tubulin isotypes in human prostate carcinoma and benign prostatic hypertrophy,” Prostate 30, 263-268, 1997) report that normal cells of the prostate do not have nuclear beta II. Cancer cells of the prostate were shown to contain beta II 100% of the time. This correlation suggests that it is possible to identify the presence of prostate cancer in a patient by sampling the blood of these patients, separating the prostate cells in the circulation, isolating the nuclei from these cells and testing for the presence of RNA for the beta II tubulin gene. Isolation of different cell types may include but are not limited to, cells of the breast, kidney, liver and bone marrow.

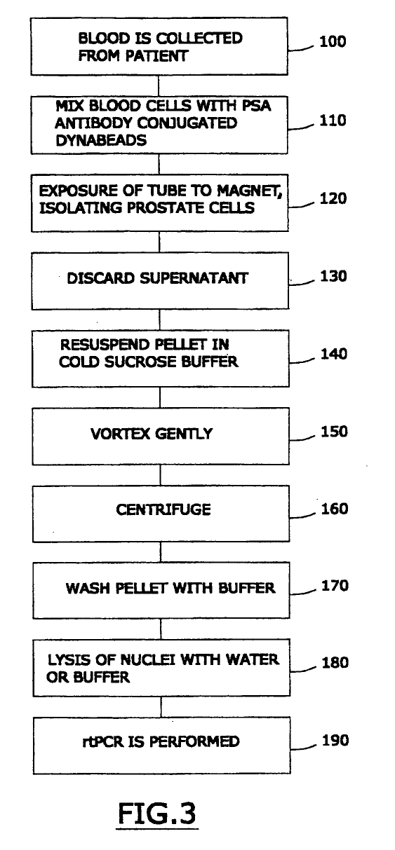

Referring to FIG. 3, and to the preferred process depicted therein, in step 100 blood is collected from a patient using methods well known to those in the art. Reference may be had, e.g., to U.S. Pat. Nos. 6,605,048 (vacuum device to assit in the drawing of capillary blood samples), 6,692,479 (donor blood sampling system), 6,719,771 (blood sampling deivce), 6,736,738 (automated blood sampling apparatus), 6,775,802 (whole blood sampling device), 6,776,959 (vessel for blood sampling), and the like. The entire disclosure of each of these United States patents is hereby incorporated by reference into this specification.

In one preferred embodiment, RNA (ribonucleic acid) is extracted from the nuclei of the cells of the blood sampled in step 100. In order to do this, one may use, and slightly modify, the process described in U.S. Pat. No. 6,329,179 (“Method enabling use of extracellular RNA extracted from plasma or serum to detect, monitor or evaluate cancer”), the entire disclosure of which is hereby incorporated by reference into this specification. This patent claims “1. A method for detecting tumor-derived or tumor-associated RNA in the plasma or serum fraction of blood from a human or animal as an aid in the detection, diagnosis, monitoring, treatment, or evaluation of neoplastic disease, including early cancer, non-invasive cancer, premalignant states, invasive cancer, advanced cancer, and benign neoplasm, wherein the tumor-derived or tumor-associated RNA is tyrosinase RNA, keratin RNA, prostate-specific antigen RNA, alpha-fetoprotein RNA, BCR/abl RNA, carcinoembryonic antigen RNA, p97 RNA, MUC 18 RNA, PML/RAR RNA, CD44 RNA, EWS/FLI-1 RNA, EWS/ERG RNA, AML1/ETO RNA, MAGE RNA, beta human chorionic gonadotropin RNA, or telomerase-associated RNA, the method comprising the steps of: a) extracting mammalian RNA from plasma or serum, wherein a fraction of said extracted heterogeneous RNA comprises a tumor-derived or tumor-specific RNA species that is tyrosinase RNA, keratin RNA, prostate-specific antigen RNA, alpha-fetoprotein RNA, BCR/abl RNA, carcinoembryonic antigen RNA, p97 RNA, MUC 18 RNA, PML/RAR RNA, CD44 RNA, EWS/FLI-1 RNA, EWS/ERG RNA, AMLI/ETO RNA, MAGE RNA, beta human chorionic gonadotropin RNA, or telomerase-associated RNA; b) amplifying or signal amplifying said fraction of the extracted RNA or corresponding cDNA, wherein amplification is performed in either a qualitative or quantitative fashion using primers or probes specific for the tumor-derived or tumor-associated RNA or corresponding cDNA; and c) detecting the amplified RNA or corresponding cDNA product, or the signal amplified product associated with the RNA or corresponding cDNA.”

U.S. Pat. No. 6,629,179 discloses that “Ribonucleic acid (RNA) is essential to the processes which allow translation of the genetic code to form proteins necessary for all cellular functions, both in normal and neoplastic cells. While the genetic code structurally exists as deoxyribonucleic acid (DNA), it is the function of RNA, existing as the subtypes transfer-RNA, messenger-RNA or messenger-like RNA, and ribosomal-RNA, to carry and translate this code to the cellular sites of protein production. In the nucleus, this RNA may further exist as or in association with ribonucleoproteins (RNP). The pathogenesis and regulation of cancer is dependent upon RNA-mediated translation of specific genetic codes, which often reflects mutational events within oncogenes, to produce proteins involved with cell proliferation, regulation, and death. Furthermore, other RNA and their translated proteins, although not necessarily those involved in neoplastic pathogenesis or regulation, may serve to delineate recognizable characteristics of particular neoplasms by either being elevated or inappropriately expressed. Thus, recognition of specific RNA can enable the identification, detection, inference, monitoring, or evaluation of any neoplasm, benign, malignant, or premalignant, in humans and animals. Furthermore, since RNA can be repetitively created from its DNA template, for a given gene within a cell there may be formed a substantially greater number of associated RNA molecules than DNA molecules. Thus, an RNA-based assay should have greater sensitivity, and greater clinical utility, than its respective DNA-based assay. Note that the term RNA denotes ribonucleic acid including fragments of ribonucleic acid consisting of ribonucleic acid sequences.”

U.S. Pat. No. 6,629,179 also discloses that “RNA based nucleic acid amplification assays, including the reverse transcriptase polymerase chain reaction (RT-PCR, also known as reverse transcription polymerase chain reaction or RNA-PCR), branched DNA signal amplification, and self-sustained sequence replication assays, such as isothermal nucleic acid sequence based amplification (NASBA), have proven to be highly sensitive and specific methods for detecting small numbers of RNA molecules. As such, they can be used in direct assays of neoplastic tissue (1-3). Since peripheral blood is readily obtainable from patients with cancer, and metastatic cancer cells are known to circulate in the blood of patients with advanced cancer, several investigators have recently used RT-PCR to detect intracellular RNA extracted from circulating cancer cells (4-7). It must be emphasized that currently investigators apply RT-PCR to detect extracted intracellular RNA from a predominately cellular fraction of blood in order to demonstrate the existence of circulating cancer cells. RT-PCR is applied only to the cellular fraction of blood obtained from cancer patients, i.e., the cell pellet or cells within whole blood. The plasma or serum fraction of blood is usually discarded prior to analysis, but is not examined separately. Since such a cellular fraction approach relies upon the presence of metastatic circulating cancer cells, it is of limited clinical use in patients with early cancers, and is not useful in the detection of non-invasive neoplasms or pre-malignant states.”

U.S. Pat. No. 6,629,179 also discloses that “The invention described by this patent application demonstrates the novel use of that human or animal tumor-derived or tumor-associated RNA found circulating in the plasma or serum fraction of blood, as a means to detect, monitor, or evaluate cancer and premalignant states. This invention is based upon the application of RNA extraction techniques and nucleic acid amplification assays to detect tumor-derived or associated extracellular RNA found circulating in plasma or serum. In contrast to the detection of viral-related RNA in plasma or serum, and the detection of tumor-associated DNA in plasma and serum, the detection of human or mammalian RNA, and particularly tumor-derived or associated RNA, has never been detected specifically within the plasma or serum fraction of blood using nucleic acid amplification methodology, and thus represents a novel and non-obvious use for these RNA extraction methods and nucleic acid amplification assays. Since this invention is not dependent upon the presence of circulating cancer cells, it is clinically applicable to cases of early cancer, non-invasive cancers, and premalignant states, in addition to cases of invasive cancer and advanced cancer. Further, this invention allows the detection of RNA in previously frozen or otherwise stored plasma and serum, thus making plasma and serum banks available for analysis and otherwise increasing general usefulness.”

U.S. Pat. No. 6,629,179 also discloses that “Tumor-derived or tumor-associated RNA that is present in plasma and serum may exist in two forms. The first being extracellular RNA, but the second being extractable intracellular RNA from cells occasionally contaminating the plasma or serum fraction. In practice, it is not necessary to differentiate between intracellular and extracellular in order to detect RNA in plasma or serum using the invention, and this invention can be used for detection of both. The potential uses of tumor-derived or associated extracellular RNA have not been obvious to the scientific community, nor has the application of nucleic acid amplification assays to detect tumor-derived or associated extracellular RNA been obvious. Indeed, the very existence of tumor-derived or associated extracellular RNA has not been obvious to the scientific community, and is generally considered not to exist. It is generally believed that plasma ribonucleases rapidly degrade any extracellular mammalian RNA which might circulate in blood, rendering it nondetectable (8). Komeda et al., for example, specifically added free RNA to whole blood obtained from normal volunteers, but were unable to detect that RNA using PCR (54). However, nucleases appear inhibited in the plasma of cancer patients (9). In addition, extracellular RNA, either complexed to lipids and proteolipids, protein-bound, or within apoptotic bodies, would be protected from ribonucleases. Thus, although still undefined, tumor-derived or associated extracellular RNA may be present in plasma or serum via several mechanisms. Extracellular RNA could be secreted or shed from tumor in the form of lipoprotein (proteo-lipid)-RNA or lipid-RNA complexes, it could be found within circulating apoptotic bodies derived from apoptotic tumor cells, it could be found in proteo-RNA complexes released from viable or dying cells including or in association with ribonucleoproteins, or in association with other proteins such as galectin-3, or RNA could be released from necrotic cells and then circulate bound to proteins normally present in plasma. Additionally it could exist circulating within RNA-DNA complexes including those associated with ribonucleoproteins and other nucleic RNA. Further, RNA may exist within several of these moieties simultaneously. For example, RNA may be found associated with ribonucleoprotein found within proteo-lipid apoptotic bodies. The presence of extracellular RNA in plasma or serum makes their detection by nucleic acid amplification assays feasible.”

U.S. Pat. No. 6,629,179 also discloses that “Several studies in the literature support the existence of tumor-derived or associated extracellular RNA. RNA has been shown to be present on the cell surface of tumor cells, as demonstrated by electrophoresis (10), membrane preparations (11), and P32 release (12). Shedding of phospholipid vesicles from tumor cells is a well described phenomena (13, 14), and similar vesicles have been shown to circulate in the blood of patients with cancer (15). Kamm and Smith used a fluorometric method to quantitate RNA concentrations in the plasma of healthy individuals (55). Rosi and colleagues used high resolution nuclear magnetic resonance (NMR) spectroscopy to demonstrate RNA molecules complexed with lipid vesicles which were shed from a human colon adenocarcinoma cell line (16). Further characterization of these lipid-RNA complexes demonstrated the vesicles additionally contained triglycerides, cholesterol esters, lipids, oligopeptide, and phospholipids (17). Mountford et al. used magnetic resonance spectroscopy to identify a proteolipid in the plasma of a patient with an ovarian neoplasm (18). While further evaluation of the proteolipid using the orcinol method suggested RNA was present, this could not be confirmed using other methods. Wieczorek and associates, using UV spectrometry and hydrolysis by RNases, claimed to have found a specific RNA-proteolipid complex in the serum of cancer patients which was not present in healthy individuals (19-20). The complex had unvarying composition regardless of the type cancer. Wieczorek et al. were further able to detect this specific RNA-proteolipid complex using a phage DNA cloned into E. Coli and hybridized to RNA from neoplastic serum, a method distinctly different from the method of this invention. The DNA was then detected by immunoassay (21). However, the RNA found in this complex is described as 10 kilobases, which is so large as to make it questionable whether this truly represents RNA as described. More recently, DNA and RNA-containing nucleoprotein complexes, possibly representing functional nuclear suborganellular elements, were isolated from the nuclei of lymphoma cells (22). It was not shown, however, that these complexes can be shed extracellularly. Other ribonucleoprotein complexes have been associated with c-myc oncogene RNA (56).”

Referring again to FIG. 3, the steps 110 et seq. are a modification of the process of U.S. Pat. No. 6,329,179; using this modification, it is possible to isolate and identify the presence of nuclear beta II in cells in the blood.

In step 110, and referring again to FIG. 3, the blood cells collected in step 100 are mixed with PSA (prostate specific antigen) antibody conjugated dynabeads in order to isolate circulating prostate cells from whole blood.

In the embodiment depicted for step 110, one may use commercially available products such as, e.g., Dynabeads. A summary of how this product is used is summarized in the promotion material of the Dynal Biotech Inc. website (at http://www.dynal.no/kunder/dynal/dynalpub401.nsf/$all/786DDD00C03AC92CC1256E200053 D887?open&qm=TopLeftMenu,1,3,0) which states: “Dynabeads are pre-coated with antibodies against specific cell surface antigens—or you can add your own. Add the appropriate Dynabeads for the target cell to your starting sample (whole or cord blood, bone marrow, buffy coat, mononuclear cell (MNC), mouse lymph node or spleen). Once the beads have captured the cells, place the tube in a convenient magnet, a Dynal MPC. Bound cells are quickly pulled to the tube wall and the supernatant can be transferred to a new tube or discarded, depending on your chosen method (shown in FIG. 5, step 520).” More specifically, “Any cell source may be used, even viscous samples such as whole blood, buffy coat, bone marrow or digested tissue samples. After a short incubation, Dynabeads bind to the target cells and the bead-captured cells are easily separated from the crude sample in less than 2 minutes by applying a magnet to the tube.”

Dynabeads, their manufacture, the protocols for their use and modification are described in several United States, the entire disclosure of which is hereby incorporated by reference into this specification. Reference may be had, e.g., to U.S. Pat. Nos. 5,512,439 (“Nucleic Acid Probes”), 5,759,820 (“Process for producing cDNA”). 5,962,223 (“Method of enriching rare cells), 6,265,229 (Method and deivce for detection of specific target cells in specialized or mixed cell population”), 6,632,620 (“Compositions for the identification and isolation of stem cells”), 6,767,635 (“Magentic nanoparticles having biochemical activity), 6,790,604 (Method for indentifying carcinoma cells with metastatic potential), and the like. The entire disclosure of each of these United States patents is hereby incorporated by reference into this specification.

U.S. Pat. No. 6,767,635 is of interest in another regard, claiming (in claim 1)” 1. Magnetic nanoparticles having biochemical activity, consisting of a magnetic core particle and an envelope layer fixed to the core particle, wherein the magnetic nanoparticles comprise a compound of general formula M-S-L-Z (I), the linkage sites between S and L and, L and Z further comprise covalently bound functional groups, wherein M represents said magnetic core particle; S represents a biocompatible substrate fixed to M; L represents a linker group, and Z represents a group comprised of nucleic acids, peptides or proteins or derivatives thereof, at least one of which binds to an intracellularomacromolecule.” The specification of U.S. Pat. No. 6,767,635 discloses that “The most frequent causes of death include cancers. In particular, more and more people die from lung, breast and prostate cancers. Presently, the primary objectives of medicine therefore include the control of cancers.”

Referring again to FIG. 3, the company materials that relate to Dynabeads cover “immobilisation of any target nucleic acid through hybridisation with an oligonucleotide probe linked directly or indirectly to Dynabeads®-like particles. These patents also cover the use of particle-linked oligonucleotide as primer for cDNA synthesis, as well as methods for subtraction cloning, in vitro mutagenesis and quantification of target through synthesis labelled cDNA.”

Referring again to FIG. 3, and to step 110 thereof, Prostate Specific Antigen (PSA) is a glycoprotein found exclusively on the surface of cells of the prostate. Reference may be had, e.g., to United States patents Attachment of antibodies specific to PSA to the Dynabeads, as is accomplished by using published protocols, would allow for isolation of the prostate cells from whole blood, using protocols described above by a person of ordinary skill in the art. Antibodies to the PSA protein appropriate for attachment to the Dynabeads are commercially available from Research Diagnostics Inc (Pleasant Hill Road Flanders N.J. 07836 USA) (catalog number: #RDI-PRO10815) or from Serotec Inc. (3200 Atlantic Avenue Raleigh, N.C. 27604) (catalog number: MCA1916HT) or from HyTest Ltd. Pharmacity, 5th floor Itainen Pitkakatu 4C 20520 Turku, Finland (catalog number: 4P33). Additionally, antibodies for other tissue cell surface specific antigen including, but not limited to breast, kidney, and liver, can be obtained from these or other vendors with such products.

Referring again to FIG. 3, and in step 120 thereof, the antibody-conjugated Dynabeads are then subjected to a magnetic fields to isolate the prostate cells, which are non-magnetic. Thereafter, in step 130, the non-prostate cells and other blood components are discarded, leaving isolated prostate cells as a pellet. Thereafter, the nuclei of the isolated prostate cells are separated from the cells by conventional means such as, e.g., the means disclosed in steps 140-170. The step 130 may use the isolation protocol described in the protocol handbook of the “NCI ETI Branch Flow Cytometry Core Laboratory” (disclosed at http://home.ncifcrf.gov/ccr/flowcore/nuclei.htm).

In step 140, the pellet is resuspended in a cold sucrose buffer, as is taught in such protocol, which states that “cells are then resuspended in cold nuclei extraction buffer (320 mM sucrose, 5 mM MgCl2, 10 mM HEPES, 1% Triton X-100 at pH 7.4) at approximately 1 ml per 1 million cells.”

Thereafter, in step 150, and further quoting such protocol, “The cells are gently vortexed for 10 seconds and allowed to incubate on ice for 10 minutes.” Thereafter, in step 160 (and also quoting from such protocol) “No dounce homogenization is necessary. Nuclei were then pelleted by centrifugation at 2000×g and washed twice with nuclei wash buffer (320 mM sucrose, 5 mM MgCl2, 10 mM HEPES at pH 7.4, no Triton X-100).” As will be apparent, the wasghin step is step 170.

Referring again to FIG. 3, and in step 180 thereof, and again quoting from such protocol, “Nuclei yield and integrity were confirmed by microscopic examination with trypan blue staining. For all cell types tested (mainly connective and lymphoid tissues), this extraction procedure gives greater than 98% successful lysis with little debris and minimal clumping.” Reference also may be had to step 190 of FIG. 3.

Alternatively, or additionally, isolation of this nucleus from these prostate cells can also be accomplished using methods disclosed in WO9953025A1 (“Method of nuclear transfer”), which is hereby incorporated in its entirety by reference into this specification. This patent discloses that “The nucleus may be isolated from the donor cell by rupturing the plasma membrane of the donor cell and separating the intact nucleus from the plasma membrane and at least some of the cytoplasmic material. Following this isolation step the nucleus may still be associated with some of the cytoplasmic material of the donor cell. Preferably the nucleus is isolated from the donor cell by drawing the donor cell into a tube, whereby the plasma membrane of the cell ruptures and the intact nucleus is drawn into the tube, optionally together with some of the cytoplasmic material of the donor cell. The tube may have an internal diameter substantially less than that of the donor cell and approximately the same or greater than that of the nucleus. Preferably the tube has an internal diameter of between approximately 4 and 8 micrometers, more preferably between approximately 4 and 5 micrometers. Preferably the tube is a pipette, more preferably a micromanipulation pipette, most preferably a glass micromanipulation pipette. Preferably the donor cell is placed in a viscous solution, for example methylcellulose, for the nucleus isolation procedure.”

As discussed hereinabove, blood tests looking for the presence of cancer causing “oncogenes” have been described for the different cell types (De Luca, A., et al. “Detection of Circulating Tumor Cells in Carcinoma Patients by a Novel Epidermal Growth Factor Receptor Reverse Transcription-PCR Assay” Clin. Cancer Res. Vol. 6, 1439-1444, April 2000); the property described in this disclosure, a physical property of the cell (namely the existence of beta II in the nucleus) is a more powerful diagnostic test for the reasons that, first, cells can express oncogenes and not be cancerous. To quote, again, the authors of U.S. Pat. No. 6,329,179, “While not related to the claims of this patent, similar analogy likely exists for detection of normal RNA (non-tumor derived or non-tumor associated RNA) in plasma and serum. Subsequent to the filing of the provisional patent application for this patent, the inventor has shown that normal RNA (non-tumor derived RNA) could similarly be detected in the plasma or serum of both healthy volunteers and cancer patients using extraction methods and amplification methods as described by this invention.” However, such United States patent also discloses that “qualitative results suggested that amplified product was greater when obtained from cancer patients. Further, use of a 0.5 micron filter prior to amplification reduced, but did not eliminate amplifiable RNA, consistent with extracellular RNA being of variable size, with additional contaminating cells possible.” This may mean that simply relying on the detection of oncogene RNA in a blood sample may mislead a clinician to the false assumption that cancer is present when, in fact, it is not. Additionally, as in the critique of the PSA test quoted above, a test in which the amount of the antigen detected is the focus of the assay, a relative measure for each patient must be known. Third, many individual genes can contribute cell transformation. Looking for all possible genes or combinations of genes that contribute to a cancer cell's transformation is not technically achievable.

Referring again to FIG. 5, and in step 180 thereof, the cell nuclei can be lysed by the resuspension of the nuclei pellet in cold water containing Rnase inhibitor, which will be well understood but those with ordinary skill in the art. rtPCR can be performed on this lysed nuclei solution (FIG. 5, step 590) using standard methods like the one disclosed below in U.S. Pat. No. 5,688,649, “Methods of detecting micrometastasis of prostate cancer,” the entire disclosure of which is hereby incorporated by reference into this specification.

U.S. Pat. No. 5,688,649 claims “1. A method of detecting prostate cancer micrometastasis in a patient comprising the steps of: obtaining a sample of blood of the patient; obtaining a sample of RNA from the blood sample wherein said RNA is obtained from cells from the buffy coat of the Ficoll gradient of a prepared blood sample; detecting the presence of RNA that encodes prostate specific antigen in said sample of RNA; wherein said presence of RNA that encodes prostate specific antigen indicates circulating hematogenous micrometastasis of prostate cancer.” This patent discloses that “In accordance with the present invention, methods of detecting prostate cancer micrometastasis in a patient are provided comprising the steps of obtaining a sample of RNA from a patient's blood and amplifying said RNA with polymerase chain reaction. The polymerase chain reaction is performed using a pair of primers which are complementary to separate regions of the prostate specific antigen gene.”

The process of U.S. Pat. No. 5,688,649, and the processs of U.S. Pat. No. 6,329,179 do not differentiate between cellular and extracellular RNA in the circulation. Neither address the separation of nucleus from the rest of the cell. Further, U.S. Pat. No. 5,688,649 discloses a method for recovering RNA from whole cells which are removed from blood that is accomplished thus: “In accordance with methods of the present invention, methods of detecting micrometastasis of prostate cancer in a patient is provided comprising the step of obtaining a sample of RNA from the patient's blood. Preferably the RNA is obtained from a blood sample such as a peripheral venous blood sample. A whole blood gradient may be performed to isolate nucleated cells and total RNA is extracted such as by the RNazole B method (Tel-Test Inc., Friendswood, Tex.) or by modification of methods known in the art such as described in Sambrook et al., Molecular Cloning: A Laboratory Manual (Cold Spring Harbor Laboratory, Cold Spring Harbor, N.Y., 1989). Thereafter, a polymerase chain reaction may be performed on the total extracted RNA. Preferably a reverse transcriptase PCR amplification procedure may be performed in order to quantify the amount of mRNA amplified. Polymerase chain reaction methodologies are well known in the art. Innis et al., PCR Protocols, Academic Press, Inc., San Diego Calif., 1990. Polymerase chain reaction primers may be designed to be complementary to separate regions of the prostate specific antigen (PSA) gene. Henttu et al., Biochem. Biophys. Res. Comm. 1989, 160, 903-910.” These same methods work well as disclosed, or slightly modified, to gather data from the RNA contained in the cell nucleus.

It is apparent from the above that many modifications and changes are possible without departing from the spirit and scope of the present invention.

1. An anti-mitotic composition with a therapeutic index greater than 1.1, a clonogenic survival rate of less than 0.1 percent, and a binding affinity index for a beta tubulin isotype selected from the group consisting of the class II isotype, the class III isotype, and the class V isotype. one at least about 1.1.

2. The anti-mitotic composition as recited in claim 1, wherein said anti-mitotic composition is comprised of a first antirnitotic agent and a second anti-mitotic agent.

3. The anti-mitotic composition as recited in claim 2, wherein each of said first anti-mitotic composition and said second anti-mitotic composition has an mitotic factor index of at least about 10 percent.

4. The anti-mitotic composition as recited in claim 2, wherein each of said first anti-mitotic composition and said second anti-mitotic composition has an mitotic factor index of at least about 30 percent.

5. The anti-mitotic composition as recited in claim 2, wherein each of said first anti-mitotic composition and said second anti-mitotic composition has an mitotic factor index of at least about 50 percent.

6. The anti-mitotic composition as recited in claim 2, wherein said first anti-mitotic agent has a suppressivity of at least 1,000.

7. The anti-mitotic composition as recited in claim 2, wherein said composition is comprised of a thioredoxin system inhibitor.

8. The anti-mitotic composition as recited in claim 7, wherein said first anti-mitotic agent is a thioredoxin system inhibitor.

9. The anti-mitotic composition as recited in claim 7, wherein said thioredoxin system inhibitor is irofulven.

10. The anti-mitotic composition as recited in claim 2, wherein said beta-tubulin isotype is the class II isotype.

11. The anti-mitotic composition as recited in claim 2, wherein said beta-tubulin isotype is the class III isotype.

12. The anti-mitotic composition as recited in claim 2, wherein said beta-tubulin isotype is the class V isotype.

13. The anti-mitotic composition as recited in daim 2, wherein said beta-tubulin isotype is the class II isotype.

14. The anti-mitotic composition as recited in claim 2, wherein said composition has a therapeutic index of at least about 2.

15. The anti-mitotic composition as recited in claim 2, wherein said composition has a therapeutic index of at least about 5.

16. The anft-mitotic composition as recited in claim 2, wherein said composition has a therapeutic index of at least about 10.

17. The anti-mitotic composition as recited in claim 2, said first anti-mitotic agent, with regard to a first tubulin dimer selected from the group consisting the alpha/beta II dimer, the alpha/beta III dimer, and the and alpha/beta V dimer, has K_{d1} value that is at least 1.1 times as great as the K_{d1} value for unfractionated tubulin.

18. The anti-mitotic composition as recited in claim 2, said first anti-mitotic agent, with regard to a first tubulin dimer selected from the group consisting the alpha/beta II dimer, the alpha/beta III dimer, and the and alpha/beta V dimer, has K_{d1} value that is at least 1.5 times as great as the K_{d1} value for unfractionated tubulin.

19. The anti-mitotic composition as recited in claim 18 wherein said first anti-mitotic agent, with regard to a first tubulin dimer selected from the group consisting the alpha/beta II dimer, the alpha/beta III dimer, and the and alpha/beta V dimer, has K_{d1} value that is at least 1.5 times as great as the highest K_{d1} value for a dimer selected from the group consisting the alpha/beta I dimer, the alpha/beta IV dimer, and the and alpha/beta VI dimer.

20. The anti-mitotic composition as recited in claim 2, wherein the therapeutic index of said anti-mitotic composition is lower than the therapeutic index of said first anti-mitotic agent, and is also lower than the therapeutic index of said second anti-mitotic index.
